SEQUENCE LISTING (1 sequence)

SEQ ID NO: 1 - protein, 12 aa, Artificial sequence
  Synthetic peptide
GRRRRRRRRGDS

(19) United States
(12) Patent Application Publication
(10) Pub. No.: US 2017/0202783 A1
(43) Pub. Date: Jul. 20, 2017

(54) Amphiphilic Peptide Nanoparticles for Use as Hydrophobic Drug Carriers and Antibacterial Agents

(71) Applicant: Northeastern University, Boston, MA (US)

(72) Inventors: Run CHANG, Brighton, MA (US); Linlin SUN, Dorchester, MA (US); Thomas Jay WEBSTER, Barrington, RI (US); Gujie MI, Malden, MA (US)

(21) Appl. No.: 15/324,158

(22) PCT Filed: Jul. 8, 2015
(86) PCT No.: PCT/US15/39599

§ 371 Date: Jan. 5, 2017

Related U.S. Application Data
(60) Provisional application No. 62/021,857, filed on Jul. 8, 2014.

(51) Int. Cl. A61K 9/51; A61L 27/22; A61K 8/64; A61L 27/34; A61K 31/12; A61Q 17/00; A61K 8/11; A61L 27/40

(52) U.S. Cl. CPC A61K 9/5169; A61K 8/11; A61L 27/227; A61L 27/40; A61L 27/34; A61K 31/12; A61Q 17/005; A61K 8/64; A61L 2400/18

(57) ABSTRACT

Nanoparticulate carrier formulations are useful to solubilize, deliver, and target hydrophobic drugs for treating diseases including cancer and bacterial infections. The formulations contain amphiphilic peptides having a hydrophobic portion and a positively charged hydrophilic portion. The peptides self-associate at nonacidic pH to form mi-celles with a spherical nanoparticle morphology. The hydrophobic core of the nano-particles encapsulates hydrophobic drugs, including antitumor agents, increasing their solubility in water and allowing them to be targeted, for example, to cancer cells. The positively charged surface of the nanoparticles, together with an optional targeting moiety such as an RGD peptide, allows the nanoparticles to bind selectively to mammalian cells and bacterial cells, including cancer cells that overexpress integrin receptors. The pH-dependence of the nanoparticle association/dissociation can be employed to conveniently load the nanoparticles with hydrophobic drug using a controlled pH shift, and unload them in acidic intracellular compartments. The ability of the carrier formulations to solubilize and target hydrophobic drugs gives rise to strategies for the selective inhibition or killing of cancer cells, such as the killing of osteosarcoma cells using the drug curcumin. The amphiphilic peptides and nanoparticles derived therefrom also give rise to additional compositions and methods that have useful bacteriocidal features as well as the ability to promote cell adhesion in cell scaffolds and coatings for medical implants.

### BACKGROUND

Curcumin is an example of a hydrophobic drug that is difficult to administer and deliver to its target because of its insolubility. It has potential as a chemotherapeutic agent in many types of cancer since it possesses pleiotropic anticarcinogenesis effects. Curcumin targets several cellular processes including gene expression, transcription, proliferation, and extracellular matrix synthesis.¹ Curcumin not only shows antiproliferative effects towards many types of cancer by inhibiting NF-kB and its downstream gene products, but also affects various growth receptors and cell adhesion molecules involved in tumor growth.²⁻⁴ In addition, curcumin has been shown to upregulate p53 expression in various cancer cell lines, including osteosarcoma cells.⁵⁻⁷ However, with its polyphenol structure, curcumin is insoluble in water.⁸ Curcumin is unstable in alkaline conditions and has a high degradation rate under physiological conditions, e.g., in phosphate buffers at pH 7.2.⁹

There remains a need to develop suitable carriers for the administration of curcumin and other hydrophobic drugs.

### SUMMARY OF THE INVENTION

The invention provides nanoparticulate carrier formulations for hydrophobic drugs and methods related to their production and use in treating diseases including cancer and bacterial infections. Amphiphilic peptides containing a hydrophobic portion and a positively charged hydrophilic portion self-associate at nonacidic pH to form micelles with a spherical nanoparticle morphology. The hydrophobic core of the nanoparticles can be used to encapsulate or embed hydrophobic drugs, including antitumor agents. The positively charged surface of the nanoparticles, together with an optional targeting moiety such as an RGD peptide, allows the nanoparticles to bind selectively to mammalian cells and bacterial cells, including cancer cells that overexpress integrin receptors. Because the nanoparticles reversibly dissociate at low pH, they can deliver the encapsulated or embedded hydrophobic drug into the interior of target cells. The pH-dependence of the nanoparticle association/dissociation can be employed to conveniently load the nanoparticles with hydrophobic drug using a controlled pH shift. The ability of the carrier formulations to solubilize and target hydrophobic drugs gives rise to strategies for the selective inhibition or killing of cancer cells, such as the killing of osteosarcoma cells using the drug curcumin. The amphiphilic peptides and nanoparticles derived therefrom also give rise to additional compositions and methods that have useful bacteriocidal features as well as the ability to promote cell adhesion in cell scaffolds and coatings for medical implants.

One aspect of the invention is a nanoparticulate carrier formulation for a hydrophobic drug. The formulation includes a plurality of amphiphilic peptide molecules and a plurality of hydrophobic drug molecules. Each peptide molecule contains a hydrophobic portion covalently linked to a positively charged hydrophilic portion. The molecules are assembled into a plurality of substantially spherical nanoparticles in an aqueous medium having a nonacidic pH, with each nanoparticle having a hydrophobic core. The hydrophobic drug molecules are embedded in the hydrophobic core of the nanoparticles. The hydrophobic drug is thereby solubilized in the aqueous medium of the formulation at a higher concentration than the solubility limit of the hydrophobic drug alone in the aqueous medium. The nanoparticles are capable of delivering the drug to the interior of a mammalian cell.

Another aspect of the invention is a method of making the nanoparticulate carrier formulation described above. The method includes the steps of: (a) providing an aqueous medium having an acidic pH and containing a positively charged amphiphilic peptide in a dissociated state; (b) adding a hydrophobic drug to the aqueous medium; and (c) raising the pH of the aqueous medium. When the pH of the medium is raised, the amphiphilic peptide forms nanoparticles having a hydrophobic core, which encapsulates the hydrophobic drug or causes it to becomes embedded in the hydrophobic core of the nanoparticles.

Still another aspect of the invention is a method of administering a hydrophobic drug. The method includes administering to a subject in need thereof the nanoparticulate carrier formulation described above. After administration, the hydrophobic drug is delivered by the nanoparticle carriers to an intracellular site in the subject.

Yet another aspect of the invention is a method of inhibiting the growth and/or replication of bacteria. The method includes contacting the bacteria with a plurality of amphiphilic nanoparticles. The amphiphilic nanoparticles contain a plurality of associated amphiphilic peptide molecules, each peptide molecule including a hydrophobic portion covalently linked to a positively charged hydrophilic portion. The nanoparticles are substantially spherical and have a positively charged surface and a hydrophobic core. The nanoparticles are formulated in an aqueous medium having a nonacidic pH. Following contacting the nanoparticles with the bacteria, the growth and/or replication of the bacteria are inhibited. A related aspect is a method of treating a bacterial infection. In that method, a plurality of amphiphilic nanoparticles as described in this paragraph are administered to a subject in need thereof.

Even another aspect of the invention is a cosmetic composition capable of inhibiting the growth or replication of bacteria in or on the skin of a subject. The composition contains a plurality of amphiphilic nanoparticles. The amphiphilic nanoparticles in turn contain a plurality of associated amphiphilic peptide molecules, each having a hydrophobic portion covalently linked to a positively charged hydrophilic portion. The nanoparticles are substantially spherical and have a positively charged surface and a hydrophobic core. The composition is formulated in an aqueous medium having a nonacidic pH.

Still another aspect of the invention is a substrate for cell attachment. The substrate contains an association of amphiphilic peptide molecules, each having a hydrophobic portion covalently linked to a positively charged hydrophilic portion. The molecules are assembled into a matrix of the substrate. The hydrophobic portions of the peptide molecules are associated with each other, and the hydrophilic portions of the peptide molecules are associated with each other in the matrix. A related aspect of the invention is a medical implant which includes the cell attachment-promoting substrate, such as in a coating of the implant.

Further aspects of the invention are summarized in the following list of items:
1. A nanoparticulate carrier formulation for a hydrophobic drug, the formulation comprising
   a plurality of amphiphilic peptide molecules, each molecule comprising a hydrophobic portion covalently linked to a positively charged hydrophilic portion; wherein the molecules are assembled into a plurality of substantially spherical nanoparticles in an aqueous medium having a nonacidic pH; each nanoparticle comprising a hydrophobic core; and
   a plurality of hydrophobic drug molecules embedded in the hydrophobic core of the nanoparticles;
wherein the hydrophobic drug is solubilized in the aqueous medium of the formulation at a higher concentration than a solubility limit of the hydrophobic drug alone in the aqueous medium; and
wherein the nanoparticles are capable of delivering the drug to the interior of a mammalian cell.
2. The nanoparticulate carrier formulation of item 1, wherein said nonacidic pH is greater than about 4.
3. The nanoparticulate carrier formulation of item 2, of wherein the nanoparticles reversibly dissociate at a pH of about 4 or less and assemble at a pH greater than about 4.
4. The nanoparticulate carrier formulation of any of the preceding items, wherein the molar ratio of amphiphilic peptide molecules to hydrophobic drug molecules is from about 2:1 to about 10:1.
5. The nanoparticulate carrier formulation of any of the preceding items, wherein the hydrophobic portion comprises one or more straight or branched chain alkyl groups, cycloalkyl groups, aromatic hydrocarbons, or a combination thereof.
6. The nanoparticulate carrier formulation of item 5, wherein the hydrophobic portion comprises one or more C8 to C22 alkyl groups.
7. The nanoparticulate carrier formulation of item 6, wherein the hydrophobic portion consists of a single C18 alkyl group.
8. The nanoparticulate carrier formulation of any of the preceding items, wherein the hydrophilic portion comprises two or more amino acids capable of bearing a positive charge at a physiological pH.
9. The nanoparticulate carrier formulation of item 8, wherein the hydrophilic portion comprises five or more amino acid residues selected from arginine, lysine, and mixtures thereof.
10. The nanoparticulate carrier formulation of any of the preceding items, wherein the hydrophilic portion comprises a targeting moiety.
11. The nanoparticulate carrier formulation of item 10, wherein the targeting moiety comprises an RGD peptide, an antibody, an aptamer, or a ligand for a cell surface receptor.
12. The nanoparticulate carrier formulation of any of the preceding items, wherein the amphiphilic peptide has a log P value of 1 or more.
13. The nanoparticulate carrier formulation of any of the preceding items, wherein the amphiphilic peptide has a log D value of 1 or more at pH 7.4.
14. The nanoparticulate carrier formulation of any of the preceding items, wherein the amphiphilic peptide is C18GR7RGDS (SEQ ID NO:1).
15. The nanoparticulate carrier formulation of any of the preceding items, wherein the nanoparticles bind to a cell surface.
16. The nanoparticulate carrier formulation of any of the preceding items, wherein the nanoparticles release the hydrophobic drug molecules into an intracellular compartment having a pH of 4 or less.
17. The nanoparticulate carrier formulation of any of the preceding items, wherein the nanoparticles are taken up into a mammalian cell by micropinocytosis.
18. The nanoparticulate carrier formulation of any of the preceding items, wherein the hydrophobic drug is delivered selectively to a cancer cell.
19. The nanoparticulate carrier formulation of item 18, wherein the cancer cell is selected from the group consisting of osteosarcoma, prostate cancer, breast cancer, lung cancer, pancreatic cancer, head and neck cancer, cervical cancer, ovarian cancer, colorectal cancer, bone cancer, brain cancer, liver cancer, lymphoma, melanoma, leukemia, neuroblastoma, skin cancer, bladder cancer, uterine cancer, stomach cancer, testicular cancer, kidney cancer, intestinal cancer, throat cancer, and thyroid cancer.
20. The nanoparticulate carrier formulation of any of the preceding items, wherein the hydrophobic drug is an antitumor agent.
21. The nanoparticulate carrier formulation of any of the preceding items, wherein the hydrophobic drug is cytotoxic for a cancer cell.
22. The nanoparticulate carrier formulation of any of the preceding items, wherein the amphiphilic peptide is toxic for a bacterium or inhibits the growth or proliferation of a bacterium.
23. The nanoparticulate carrier formulation of any of the preceding items which is present in lyophilized form.
24. The nanoparticulate carrier formulation of any of the preceding items, wherein the nanoparticles have an average diameter in the range from about 10 nm to about 30 nm.
25. The nanoparticulate carrier formulation of any of the preceding items, wherein the hydrophobic drug is selected from the group consisting of curcumin, doxorubicin, paclitaxel, and cisplatin.
26. The nanoparticulate carrier formulation of any of the preceding items, wherein the hydrophobic drug has a Log P value of 1 or more.
27. The nanoparticulate carrier formulation of any of the preceding items, wherein the hydrophobic drug has a Log D value of 1 or more.
28. A method of making the nanoparticulate carrier formulation of any one of items 1-27, the method comprising the steps of:
   (a) providing an aqueous medium comprising a positively charged amphiphilic peptide, wherein the aqueous medium has an acidic pH and the amphiphilic peptide is in a dissociated state;
   (b) adding a hydrophobic drug to the aqueous medium; and
   (c) raising the pH of the aqueous medium, whereby the amphiphilic peptide forms nanoparticles having a hydrophobic core, and whereby the hydrophobic drug becomes embedded in the hydrophobic core of the nanoparticles.
29. The method of item 28, further comprising:
   (d) removing nonembedded hydrophobic drug from the aqueous suspension.
30. The method of item 29, further comprising:
   (e) lyophilizing the carrier formulation.
31. The method of any one of items 28-30, further comprising, prior to step (a):
   (a0) providing an aqueous medium comprising a positively charged amphiphilic peptide, wherein the aqueous medium has a nonacidic pH and the amphiphilic peptide is associated in the form of nanoparticles; and
   (a00) lowering the pH of the aqueous medium to an acidic pH, whereby the nanoparticles dissociate.
32. The method of any one of items 28-31, wherein step (c) comprises dialyzing the aqueous medium against a second aqueous medium having a nonacidic pH.
33. The method of any one of items 28-31, wherein steps (c) and (d) are performed simultaneously by dialyzing the aqueous medium against a second aqueous medium having a nonacidic pH and substantially lacking the hydrophobic drug.
34. The method of any one of items 28-33, wherein the pH is raised in step (c) to greater than about 4.
35. The method of item 34, wherein the pH is raised in step (c) to a value in the range from about 7.0 to about 7.4.
36. The method of any one of items 28-35, wherein the molar ratio of amphiphilic peptide molecules to hydrophobic drug molecules in the nanoparticles produced in step (c) is from about 2:1 to about 10:1.
37. The method of any one of items 28-36, wherein the amphiphilic peptide comprises a hydrophobic portion and a positively charged hydrophilic portion.
38. The method of item 37, wherein the hydrophobic portion comprises one or more straight or branched chain alkyl groups, cycloalkyl groups, aromatic hydrocarbons, or a combination thereof.
39. The method of item 38, wherein the hydrophobic portion comprises one or more C8 to C22 alkyl groups.
40. The method of item 39, wherein the hydrophobic portion consists of a single C18 alkyl group.
41. The method of item 37, wherein the hydrophilic portion comprises two or more amino acids capable of bearing a positive charge at a physiological pH.
42. The method of item 41, wherein the hydrophilic portion comprises five or more amino acid residues selected from arginine, lysine, and mixtures thereof.
43. The method of item 37, wherein the hydrophilic portion comprises a targeting moiety.
44. The method of item 43, wherein the targeting moiety comprises an RGD peptide, an antibody, an aptamer, or a ligand for a cell surface receptor.
45. The method of any one of items 28-44, wherein the amphiphilic peptide has a log P value of 1 or more.
46. The method of any one of items 28-45, wherein the amphiphilic peptide has a log D value of 1 or more at pH 7.4.
47. The method of any one of items 28-46, wherein the amphiphilic peptide is C18GR7RGDS (SEQ ID NO:1).
48. The method of any one of items 28-47, wherein the hydrophobic drug is an antitumor agent.
49. The method of any one of items 28-48, wherein the amphiphilic peptide is toxic for a bacterium or inhibits the growth or proliferation of a bacterium.
50. The method of any one of items 28-49, wherein the nanoparticles formed in step (c) have an average diameter in the range from about 10 nm to about 30 nm.
51. The method of any one of items 28-50, wherein the hydrophobic drug is selected from the group consisting of curcumin, doxorubicin, paclitaxel, and cisplatin.
52. The method of any one of items 28-51, wherein the hydrophobic drug has a Log P value of 1 or more.
53. The method of any one of items 28-52, wherein the hydrophobic drug has a Log D value of 1 or more.
54. A method of administering a hydrophobic drug, the method comprising administering to a subject in need thereof the nanoparticulate carrier formulation of any one of items 1-27, whereby the hydrophobic drug is delivered to an intracellular site in the subject.
55. The method of item 54, wherein the hydrophobic drug is selectively delivered to cells of the subject in need of treatment with the hydrophobic drug.
56. The method of any one of items 54-55, wherein the subject has cancer, and the hydrophobic drug is cytotoxic for cancer cells in the subject.
57. The method of any one of items 54-56, wherein the hydrophobic drug is selected from the group consisting of curcumin, doxorubicin, paclitaxel, and cisplatin.
58. The method of item 56, wherein the cancer is selected from the group consisting of osteosarcoma, prostate cancer, breast cancer, lung cancer, pancreatic cancer, head and neck cancer, cervical cancer, ovarian cancer, colorectal cancer, bone cancer, brain cancer, liver cancer, lymphoma, melanoma, leukemia, neuroblastoma, skin cancer, bladder cancer, uterine cancer, stomach cancer, testicular cancer, kidney cancer, intestinal cancer, throat cancer, and thyroid cancer.
59. A method of treating a bacterial infection, the method comprising administering a plurality of amphiphilic nanoparticles to a subject in need thereof; wherein the amphiphilic nanoparticles comprise a plurality of associated amphiphilic peptide molecules, each peptide molecule comprising a hydrophobic portion covalently linked to a positively charged hydrophilic portion; wherein the nanoparticles are substantially spherical and have a positively charged surface and a hydrophobic core; wherein the nanoparticles are formulated in an aqueous medium having a nonacidic pH; whereby the nanoparticles kill bacteria or inhibit the growth or replication of bacteria in the subject.
60. The method of item 59, wherein the bacterial infection is a bacterial skin infection, and the amphiphilic nanoparticles are administered to skin of the subject.
61. A method of inhibiting the growth and/or replication of bacteria, the method comprising contacting the bacteria with a plurality of amphiphilic nanoparticles; wherein the amphiphilic nanoparticles comprise a plurality of associated amphiphilic peptide molecules, each peptide molecule comprising a hydrophobic portion covalently linked to a positively charged hydrophilic portion; wherein the nanoparticles are substantially spherical and have a positively charged surface and a hydrophobic core; wherein the nanoparticles are formulated in an aqueous medium having a nonacidic pH; whereby the growth and/or replication of the bacteria are inhibited.
62. The method of item 61, wherein the hydrophobic portion of the amphiphilic peptide comprises one or more straight or branched chain alkyl groups, cycloalkyl groups, aromatic hydrocarbons, or a combination thereof.
63. The method of item 62, wherein the hydrophobic portion comprises one or more C8 to C22 alkyl groups.
64. The method of item 63, wherein the hydrophobic portion consists of a single C18 alkyl group.
65. The method of item 64, wherein the hydrophilic portion comprises two or more amino acids capable of bearing a positive charge at a physiological pH.
66. The method of item 65, wherein the hydrophilic portion comprises six or more amino acid residues selected from arginine, lysine, and mixtures thereof.
67. The method of item 65, wherein the amphiphilic peptide is C18GR7RGDS (SEQ ID NO:1).
68. A cosmetic composition capable of inhibiting the growth or replication of bacteria in or on skin; wherein the composition comprises a plurality of amphiphilic nanoparticles; wherein the amphiphilic nanoparticles comprise a plurality of associated amphiphilic peptide molecules, each peptide molecule comprising a hydrophobic portion covalently linked to a positively charged hydrophilic portion; wherein the nanoparticles are substantially spherical and have a positively charged surface and a hydrophobic core; wherein the composition is formulated in an aqueous medium having a nonacidic pH.
69. The cosmetic composition of item 68, wherein the hydrophobic portion of the amphiphilic peptide comprises one or more straight or branched chain alkyl groups, cycloalkyl groups, aromatic hydrocarbons, or a combination thereof.
70. The cosmetic composition of item 69, wherein the hydrophobic portion comprises one or more C8 to C22 alkyl groups.
71. The cosmetic composition of item 70, wherein the hydrophobic portion consists of a single C18 alkyl group.
72. The cosmetic composition of item 68, wherein the hydrophilic portion comprises two or more amino acids capable of bearing a positive charge at a physiological pH.
73. The cosmetic composition of item 72, wherein the hydrophilic portion comprises six or more amino acid residues selected from arginine, lysine, and mixtures thereof.
74. The cosmetic composition of item 68, wherein the amphiphilic peptide is C18GR7RGDS (SEQ ID NO:1).
75. A matrix for cell attachment, the matrix comprising an association of amphiphilic peptide molecules, each amphiphilic peptide molecule comprising a hydrophobic portion covalently linked to a positively charged hydrophilic portion; wherein the molecules are assembled into a matrix, wherein the hydrophobic portions and the hydrophilic portions of the peptide molecules are associated in the matrix.
76. The matrix of item 75, wherein the hydrophobic portion of the amphiphilic peptide comprises one or more straight or branched chain alkyl groups, cycloalkyl groups, aromatic hydrocarbons, or a combination thereof.
77. The matrix of item 75, wherein the hydrophobic portion comprises one or more C8 to C22 alkyl groups.
78. The matrix of item 77, wherein the hydrophobic portion consists of a single C18 alkyl group.
79. The matrix of item 75, wherein the hydrophilic portion comprises two or more amino acids capable of bearing a positive charge at a physiological pH.
80. The matrix of item 79, wherein the hydrophilic portion comprises six or more amino acid residues selected from arginine, lysine, and mixtures thereof.
81. The matrix of item 80, wherein the amphiphilic peptide is C18GR7RGDS (SEQ ID NO:1).
82. A medical implant comprising the matrix of item 75.
83. The medical implant of item 82, wherein the matrix is present as a coating on a surface of the implant.

### BRIEF DESCRIPTION OF THE DRAWINGS

FIG. 1A shows the molecular structure of the amphiphilic peptide C18GR7RGDS. FIG. 1B shows a schematic representation of an amphiphilic peptide of the invention. FIG. 1C shows a schematic representation of an embodiment of an amphiphilic nanoparticle drug carrier of the invention in cross-section. FIG. 1D shows a schematic representation of a portion of a coated medical implant according to the invention in cross-section.

FIGS. 2A-2F show negative-stained TEM images of C18GR7RGDS amphiphilic peptide nanoparticles (APNPs) under different conditions. The scale bar is 100 nm, and sizes of selected individual structures are indicated. The nanoparticles were at 1.5 mg/mL in deionized water (FIG. 2A), phosphate-buffered saline pH 7.4 (FIG. 2B), in water without sonication (FIG. 2C), in acetic acid at pH 6 (FIG. 2D), in acetic acid at pH 4 (FIG. 2E), and in acetic acid at pH 2 (FIG. 2F).

FIGS. 3A and 3B show negative-stained TEM images of curcumin-loaded C18GR7RGDS APNPs. The scale bar is 100 nm, and sizes of selected individual structures are indicated.

FIG. 4 shows the results of zeta potential measurements on pure C18GR7RGDS APNPs and curcumin-loaded C18GR7RGDS APNPs.

FIG. 5 shows Fourier transform infrared spectra of (i) solid curcumin, (ii) pure C18GR7RGDS APNPs, and (iii) curcumin-loaded C18GR7RGDS APNPs.

FIG. 6 shows X-ray diffraction patterns of of solid curcumin, pure C18GR7RGDS APNPs, and curcumin-loaded C18GR7RGDS APNPs.

FIGS. 7A-7C show bright field microscopic images of normal human osteoblast cells. FIG. 7A, control; 7B, treated with 20 μM of curcumin alone in phosphate-buffered saline; and 7C, treated with 20 μM of curcumin loaded in C18GR7RGDS APNPs. FIGS. 7D-7F show bright field microscopic images of osteosarcoma cells. FIG. 7D, control; 7E, treated with 20 μM of curcumin alone in phosphate-buffered saline; and 7F, treated with 20 μM of curcumin loaded in C18GR7RGDS APNPs.

FIGS. 8A-8C show confocal microscopic images of curcumin uptake in normal human osteoblast cells. FIG. 8A, control; 8B, treated with 20 μM of curcumin in phosphate-buffered saline; and 8C, treated with 20 μM of curcumin loaded in C18GR7RGDS APNPs. FIGS. 8D-8F show confocal microscopic images of curcumin uptake in osteosarcoma cells. FIG. 8D, control; 8E, treated with 20 μM of curcumin in phosphate-buffered saline; and 8F, treated with 20 μM of curcumin loaded in C18GR7RGDS APNPs.

FIGS. 9A and 9B show the results of a cytotoxicity study of pure C18GR7RGDS APNPs to normal human osteoblasts (HOB) and osteosarcoma (OS) cells. The data are shown as the mean±standard error of the mean of n=3 (five samples per group). P-values represent significant differences between the pure APNP-treated groups and the control groups. *P,0.01.

FIGS. 10A-10D show the results of a cytotoxicity study of curcumin-loaded C18GR7RGDS APNPs compared with curcumin alone in phosphate buffered saline and curcumin alone in DMSO. The cells in FIG. 10A and 10C were osteosarcoma (OS) cells and in FIG. 10B and FIG. 10D were normal human osteoblasts (HOB). The data are expressed as cell viability in FIGS. 10A and 10B and as cell density in FIGS. 10C and 10D. Results are shown as the mean±standard error of the mean of n=3 (five samples per group). P-values represent significant differences between labeled groups with (*) the control groups, (#) the groups treated with the same concentration of plain curcumin in PBS, and (A) the groups treated by the same concentration of curcumin dissolved in DMSO. *, #, AP,0.01; **, ##, ̂̂P,0.005.

FIGS. 11A and 11B show the effect of increasing concentrations of C18GR7RGDS APNPs on viability (measured as cell density or colony count) of human dermal fibroblasts (FIG. 11A) and S. aureus bacteria (FIG. 11B).

FIG. 12 shows the effect of various concentrations of C18GR7RGDS APNPs on growth curves of S. aureus bacteria.

### DETAILED DESCRIPTION OF THE INVENTION

The inventors have discovered carrier formulations for solubilizing and targeting hydrophobic drugs, as well as methods for using the formulations to treat diseases including cancer and bacterial infections. The formulations are based on the use of amphiphilic peptides and nanostructures containing them as carriers for hydrophobic drugs or other chemical agents. The amphiphilic peptides contain or consist of a hydrophobic portion covalently linked to a positively charged hydrophilic portion. The peptides self-associate at nonacidic pH to form micelles with a spherical nanoparticle morphology. The nanoparticles have a hydrophobic core which sequesters hydrophobic drugs and a positively charged outer surface which interacts with target cells and aids in drug delivery into the cell interior by endocytosis or pinocytosis. Such nanoparticles are referred to herein as “amphiphilic peptide nanoparticles” or “APNPs”; this term can refer to nanoparticles that are either loaded with a hydrophobic drug or nanoparticles that are devoid of drug.

The use of several protonatable groups, such as arginine or lysine, in close proximity in the hydrophilic portion makes possible a reversible association/dissociation (i.e., assembly/disassembly) mechanism for the nanoparticles that is exploited for loading and unloading of the drug in methods of the invention. Moreover, the optional inclusion of a targeting moiety, such as an RGD peptide, allows the nanoparticles to bind selectively to selected target cells. The ability of the carrier formulations to solubilize and target hydrophobic drugs allows for the selective inhibition or killing of cancer cells using drugs, such as curcumin, with limited aqueous solubility, making new therapies possible. The carrier formulations also have uses independent of drug delivery, such as killing or inhibition of bacteria and promoting cell adhesion in cell scaffolds and coatings for medical implants.

Amphiphilic molecules contain one or more polar or hydrophilic moieties linked to one or more nonpolar or hydrophobic moieties. Generally, an amphiphilic molecule has a hydrophobic portion at one end of the molecule and a hydrophilic portion at the opposite end of the molecule, and the two portions are joined by a covalent bond between them. Additional portions of the molecule may be present which are not strongly hydrophobic or hydrophilic. In the present invention, amphiphilic molecules are preferably peptides consisting of L-amino acids linked by peptide bonds, with a covalently attached hydrophobic moiety at either the N-terminal or C-terminal end of the peptide. Preferably, two or more of the amino acid residues, more preferably six or more, seven or more, or eight or more, or 4-9, or 5-10, or 5-11, or 5-12, or 6-11, or 6-12, or 7-11, or 7-12, or 8-11, or 8-12 are protonatable and capable of acquiring a positive charge at a physiological pH or at an acidic pH (i.e., less than 7.0, preferably 4.0 or less). Protonatable residues can be, for example, L-arginine, or L-lysine, or mixtures thereof, or other protonatable moieties that can be integrated into a peptide. Hydrophobic interaction of the hydrophobic moieties is the main driving force for self-assembly of amphiphilic molecules to form micelles and other nanoscale structures in aqueous solution, while the hydrophilic moieties affect the morphology of micelles and interact with water and charged moieties through hydrogen bonds and electrostatic interactions. As the protonatable residues become increasingly positively charged at acidic pH, charge repulsion effects overcome the attractive hydrophobic interactions and cause the dissociation or disassembly of the nanoparticles.

The sequestration of a hydrophobic drug or other hydrophobic chemical agent in APNPs relies on the strength of hydrophobic interactions between the drug and the hydrophobic portion of the amphiphilic peptide molecules in the APNPs. While selection of suitable amphiphilic peptides, having sufficiently strong hydrophobic interactions to bind the drug, and the identification of a drug suitable for interacting hydrophobically with the peptide molecules, can be determined empirically. For example, different combinations of amphiphilic peptides and hydrophobic drugs can be tried, and the stability of the APNPs and retention of the drug can be determined by known methods. However, theoretical approaches can also be applied. For example, peptides and drugs with suitably strong hydrophobicity can be estimated using their Log P values, determined from the equilibrium partition coefficient in an octanol/water two phase system. In order to take into account the degree of dissociation of peptides at a given pH, the related Log D values can be used. For example, a Log P value of greater than 0.8, 1.0, 1.2, 1.5, or 2.0 might be considered to represent sufficiently strong hydrophobic interactions for either the peptide or the drug. Similar values for Log D at a pH in the physiological range could indicate an acceptable ionization level. Too high an ionization level (i.e., too high a density of positive charges) can result in failure to form APNPs at required physiological pH or poor retention of the hydrophobic drug.

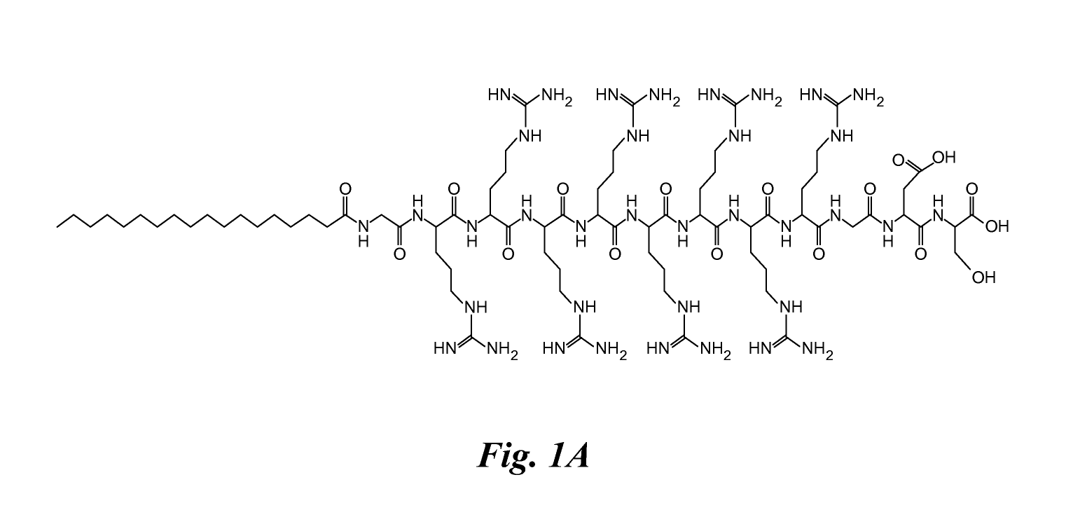

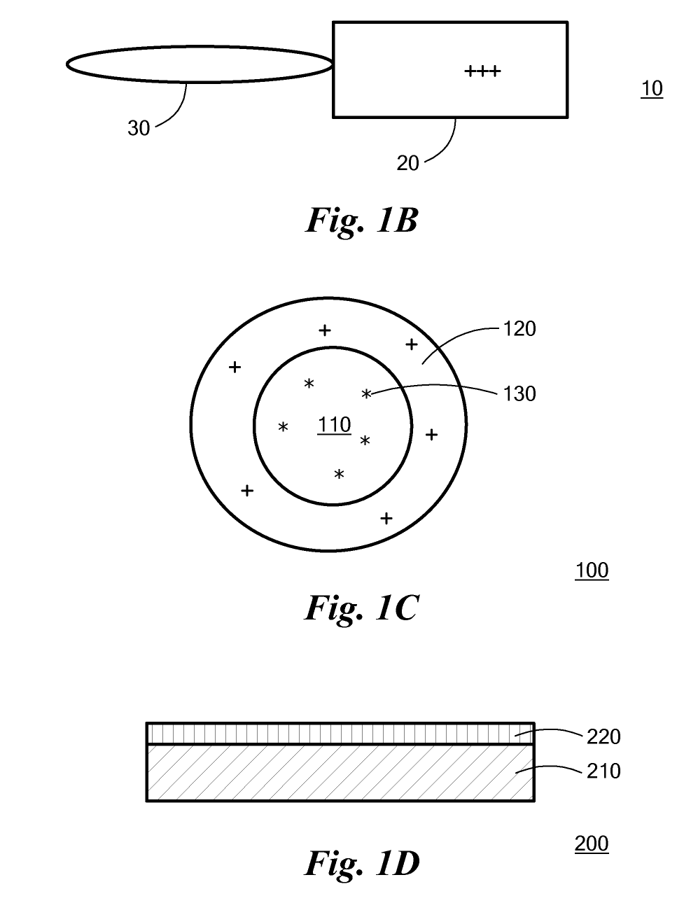

As an example, the hydrophobic drug curcumin was loaded into APNPs (see Examples 2-4). The amphiphilic peptide used was C18GR7RGDS (SEQ ID NO:1), whose structure is depicted in FIG. 1A. Since curcumin is soluble in acetic acid, curcumin was sequestered into APNP aggregates by co-dissolution of curcumin and an amphiphilic peptide with acetic acid to disrupt the previously self-assembled peptide micelle structure, followed by reforming the nanoparticles by removing the acetic acid by dialysis. Arginine deprotonation is believed to be the driving factor for this pH-sensitive self-assembly process. Although the pKa of a single arginine residue is 12.48, indicating that the guanidinium groups on the arginine-rich structure is positively charged in a physiological environment, the pKa of adjacent arginine residues is expected to be much lower due to the charge repulsion effect of adjacent positive charges. While not limiting the invention to any particular mechanism, it is believed that the dissociation of APNPs at low pH is due to the increasingly strong electrostatic repulsion as progressively more arginine residues become deprotonated at pH 4 and below, eventually leading to disruption of the nanoparticle structure.

The pH-sensitive assembly mechanism is beneficial for cellular uptake of encapsulated bioactive molecules in the inner core. For example, endosomes, in whose lumen the pH is 5-6, are membrane-bound compartments that can transport extracellular molecules from the plasma membrane to the lysosome. The lysosome can then process the molecules by digestive enzymes at a pH of about 4-5. Therefore, this low pH environment is expected to cause dissociation of APNPs and to release bioactive molecules into the cytosol.

Amphiphilic peptide molecules for use in the present invention have the general structure depicted in FIG. 1B. Amphipathic molecule 10 contains hydrophilic portion 20 linked to hydrophobic portion 30. The hydrophilic portion contains two or more protonatable groups (designated as “+++” though this is not meant to indicate an actual number of charges), which may or may not be positively charged, depending on the pH and the pKa of the individual protonatable groups. Typically, the molecule can have one or two positive charges at a physiological pH in the range from about 7.0 to about 7.4, and has more positive charges (e.g., 2-5, or up to 10, 11, or 12) at low pH (e.g., in the pH range from about 2 to about 4).

At a nonacidic pH (i.e., greater than about 4), the molecules spontaneously assemble into a micelle structure, such as that which is schematically represented in FIG. 1C. Micelle or amphipathic nanoparticle 100 contains hydrophobic core 110, which is formed by the aggregated hydrophobic portions of the amphiphilic peptide molecules, surrounded by hydrophilic shell 120, which contains a number of positive charges. The shell may also include some negative charges, but preferably has a net positive charge carried by protonatable groups, at least some of which have a pKa value in the range from about 2 to about 4, or from about 1 to about 5, or from about 1 to about 3, or from about 2 to about 5, or from about 3 to about 5. Hydrophobic drug molecules 130, if present, are located in the hydrophobic core. APNP structures such as depicted in FIG. 1C (without embedded hydrophobic drug) can be formed, for example, by simply dissolving a suitable amphiphilic peptide, such as C18GR7RGDS, in deionized water or a suitable buffer or physiological saline solution at room temperature, preferably with mixing and sonication to provide uniform and dispersed structures.

Another structure that can be formed from amphiphilic peptides of the invention is depicted in FIG. 1D. In this structure, which can be a coated medical device or implant, or a support structure for cell or tissue culture or engineering (e.g., a cell scaffold), structure 200 includes a support structure 210 upon which is deposited a matrix or coating 220 containing associated amphiphilic peptide molecules.

Surfactant-like amphiphilic peptides are amphiphiles that typically contain naturally occurring L-amino acids. Such amphiphilic peptides are biocompatible and also can be functionalized by inclusion of a variety of peptide sequences for different applications. For instance, the arginine-glycine-aspartic acid (RGD) tripeptide can target overexpressed receptors, such as αvβ3 integrins on cancer cells, while cationic peptides with 5-11 consecutive arginine residues can facilitate cellular uptake via a macropinocytosis-meditated pathway. The amphiphilic peptide, C18GR7RGDS, for example, has been used as a gene delivery carrier.¹¹

In certain embodiments the amphiphilic peptide can include a targeting moiety, which is a portion of the amphiphilic peptide, or a substituent or molecule covalently linked to the peptide, that binds to a selected target cell, such as a tumor cell. The targeting moiety may be an antibody, antibody fragment, oligonucleotide, peptide, hormone, ligand for a receptor such as a cell surface receptor, cytokine, peptidomimetic, protein, chemically modified protein, carbohydrate, chemically modified carbohydrate, chemically modified nucleic acid, or aptamer that targets a cell-surface protein. See, for example, US2011/0123451. The targeting moiety may be derived from a molecule known to bind to a cell-surface receptor. For example, the targeting moiety may be derived from low density lipoproteins, transferrin, EGF, insulin, PDGF, fibrinolytic enzymes, anti-HER2, annexins, interleukins, interferons, erythropoietins, or colony-stimulating factor. The targeting moiety may be an antibody or antibody fragment that targets the nanoparticles to the blood-brain barrier, for example, an antibody or antibody fragment to transferrin receptor, insulin receptor, IGF-I or IGF-2 receptor. See, for example, US 2002/0025313. The targeting moiety can be attached to a peptide in the nanoparticle by a linker. Linkers for coupling various moieties to peptides are known in the art.

Any hydrophobic drug or chemical agent, or any combination thereof, can be sequestered, solubilized, targeted, and/or delivered using the APNPs of the present invention. For example, the following hydrophobic drugs can be loaded into APNPs: anti-tumor agents, such as curcumin, doxorubicin, cisplatin, and paclitaxel; analgesics and anti-inflammatory agents, such as aloxiprin, auranofin, azapropazone, benorylate, diflunisal, etodolac, fenbufen, fenoprofen calcim, flurbiprofen, ibuprofen, indomethacin, ketoprofen, meclofenamic acid, mefenamic acid, nabumetone, naproxen, oxyphenbutazone, phenylbutazone, piroxicam, and sulindac; anthelmintics, such as albendazole, bephenium hydroxynaphthoate, cambendazole, dichlorophen, ivermectin, mebendazole, oxamniquine, oxfendazole, oxantel embonate, praziquantel, pyrantel embonate, and thiabendazole; anti-arrhythmic agents, such as amiodarone HCl, disopyramide, flecainide acetate, and quinidine sulphate; anti-bacterial agents, such as benethamine penicillin, cinoxacin, ciprofloxacin HCl, clarithromycin, clofazimine, cloxacillin, demeclocycline, doxycycline, erythromycin, ethionamide, imipenem, nalidixic acid, nitrofurantoin, rifampicin, spiramycin, sulphabenzamide, sulphadoxine, sulphamerazine, sulphacetamide, sulphadiazine, sulphafurazole, sulphamethoxazole, sulphapyridine, tetracycline, and trimethoprim; anti-coagulants, such as dicoumarol, dipyridamole, nicoumalone, and phenindione; anti-depressants, such as amoxapine, maprotiline HCl, mianserin HCL, nortriptyline HCl, trazodone HCL, trimipramine maleate; anti-diabetics, such as acetohexamide, chlorpropamide, glibenclamide, gliclazide, glipizide, tolazamide, tolbutamide; anti-epileptics, such as beclamide, carbamazepine, clonazepam, ethotoin, methoin, methsuximide, methylphenobarbitone, oxcarbazepine, paramethadione, phenacemide, phenobarbitone, phenytoin, phensuximide, primidone, sulthiame, and valproic acid; anti-fungal agents, such as amphotericin, butoconazole nitrate, clotrimazole, econazole nitrate, fluconazole, flucytosine, griseofulvin, itraconazole, ketoconazole, miconazole, natamycin, nystatin, sulconazole nitrate, terbinafine HCl, terconazole, tioconazole, and undecenoic acid; anti-gout agents, such as allopurinol, probenecid, and sulphin-pyrazone; anti-hypertensive agents, such as amlodipine, benidipine, darodipine, dilitazem HCl, diazoxide, felodipine, guanabenz acetate, isradipine, minoxidil, nicardipine HCl, nifedipine, nimodipine, phenoxybenzamine HCl, prazosin HCL, reserpine, and terazosin HCL; anti-malarials, such as amodiaquine, chloroquine, chlorproguanil HCl, halofantrine HCl, mefloquine HCl, proguanil HCl, pyrimethamine, and quinine sulphate; anti-migraine agents, such as dihydroergotamine mesylate, ergotamine tartrate, methysergide maleate, pizotifen maleate, and sumatriptan succinate; anti-muscarinic agents, such as atropine, benzhexol HCl, biperiden, ethopropazine HCl, hyoscyamine, mepenzolate bromide, oxyphencylcimine HCl, and tropicamide; immunosuppressants, such as aminoglutethimide, amsacrine, azathioprine, busulphan, chlorambucil, cyclosporin, dacarbazine, estramustine, etoposide, lomustine, melphalan, mercaptopurine, methotrexate, mitomycin, mitotane, mitozantrone, procarbazine HCl, tamoxifen citrate, and testolactone; anti-protazoal agents, such as benznidazole, clioquinol, decoquinate, diiodohydroxyquinoline, diloxanide furoate, dinitolmide, furzolidone, metronidazole, nimorazole, nitrofurazone, ornidazole, and tinidazole; anti-thyroid agents, such as carbimazole and propylthiouracil; anxiolytics, sedatives, hypnotics and neuroleptics, including alprazolam, amylobarbitone, barbitone, bentazepam, bromazepam, bromperidol, brotizolam, butobarbitone, carbromal, chlordiazepoxide, chlormethiazole, chlorpromazine, clobazam, clotiazepam, clozapine, diazepam, droperidol, ethinamate, flunanisone, flunitrazepam, fluopromazine, flupenthixol decanoate, fluphenazine decanoate, flurazepam, haloperidol, lorazepam, lormetazepam, medazepam, meprobamate, methaqualone, midazolam, nitrazepam, oxazepam, pentobarbitone, perphenazine pimozide, prochlorperazine, sulpiride, temazepam, thioridazine, triazolam, and zopiclone; beta-blockers, such as acebutolol, alprenolol, atenolol, labetalol, metoprolol, nadolol, oxprenolol, pindolol, and propranolol; cardiac inotropic agents, such as amrinone, digitoxin, digoxin, enoximone, lanatoside C, and medigoxin; corticosteroids, such as beclomethasone, betamethasone, budesonide, cortisone acetate, desoxymethasone, dexamethasone, fludrocortisone acetate, flunisolide, flucortolone, fluticasone propionate, hydrocortisone, methylprednisolone, prednisolone, prednisone, and triamcinolone; diuretics, such as acetazolamide, amiloride, bendrofluazide, bumetanide, chlorothiazide, chlorthalidone, ethacrynic acid, frusemide, metolazone, spironolactone, and triamterene; anti-parkinsonian agents, such as bromocriptine mesylate and lysuride maleate; gastrointestinal agents, such as bisacodyl, cimetidine, cisapride, diphenoxylate HCl, domperidone, famotidine, loperamide, mesalazine, nizatidine, omeprazole, ondansetron HCL, ranitidine HCl, and sulphasalazine; histamine receptor antagonists, such as acrivastine, astemizole, cinnarizine, cyclizine, cyproheptadine HCl, dimenhydrinate, flunarizine HCl, loratadine, meclozine HCl, oxatomide, and terfenadine; lipid regulating agents, such as bezafibrate, clofibrate, fenofibrate, gemfibrozil, probucol; nitrates and other anti-anginal agents, including amyl nitrate, glyceryl trinitrate, isosorbide dinitrate, isosorbide mononitrate, and pentaerythritol tetranitrate; nutritional supplements and vitamins, such as betacarotene, vitamin A, vitamin B.sub.2, vitamin D, vitamin E, and vitamin K; opioid analgesics, such as codeine, dextropropyoxyphene, diamorphine, dihydrocodeine, meptazinol, methadone, morphine, nalbuphine, and pentazocine; sex hormones, such as clomiphene citrate, danazol, ethinyl estradiol, medroxyprogesterone acetate, mestranol, methyltestosterone, norethisterone, norgestrel, estradiol, conjugated estrogens, progesterone, stanozolol, stibestrol, testosterone, and tibolone; and stimulants, such as amphetamine, dexamphetamine, dexfenfluramine, fenfluramine, and mazindol. See, e.g., U.S. Pat. No. 6,096,338.

The invention can be used to treat cancer by selectively targeting cancer cells with cytotoxic or anti-tumor agents. Any cancer can be targeted, including for example, prostate cancer, breast cancer, lung cancer, pancreatic cancer, head and neck cancer, cervical cancer, ovarian cancer, colorectal cancer, bone cancer, brain cancer, liver cancer, lymphoma, melanoma, leukemia, neuroblastoma, skin cancer, bladder cancer, uterine cancer, stomach cancer, testicular cancer, kidney cancer, intestinal cancer, throat cancer, and thyroid cancer.

### EXAMPLES

### Example 1

### Preparation of Amphphilic Peptide Nanoparticles

Curcumin (diferuloylmethane), acetic acid, and dimethyl sulfoxide (DMSO) were supplied by Sigma-Aldrich (St Louis, Mo., USA). The amphiphilic peptide C18GR7RGDS (molecular weight 1,850.28 g/mole) was obtained as a dry powder from Biomatik (Wilmington, Del., USA). The PlusOne Mini Dialysis Kit (molecular weight cutoff 1 kDa) was purchased from GE Healthcare (Buckinghamshire, UK).

Amphiphilic peptide nanoparticles (APNPs) were prepared by dissolving dry powder of C18GR7RGDS (FIG. 1) in deionized water followed by sonication for 60 seconds. In some experiments, the amphiphilic peptide was suspended in phosphate-buffered saline and/or acetic acid solutions at pH 2, 4, and 6. The self-assembly behavior of APNPs in these different solutions by dialysis against deionized water were then observed using a transmission electron microscope (TEM).

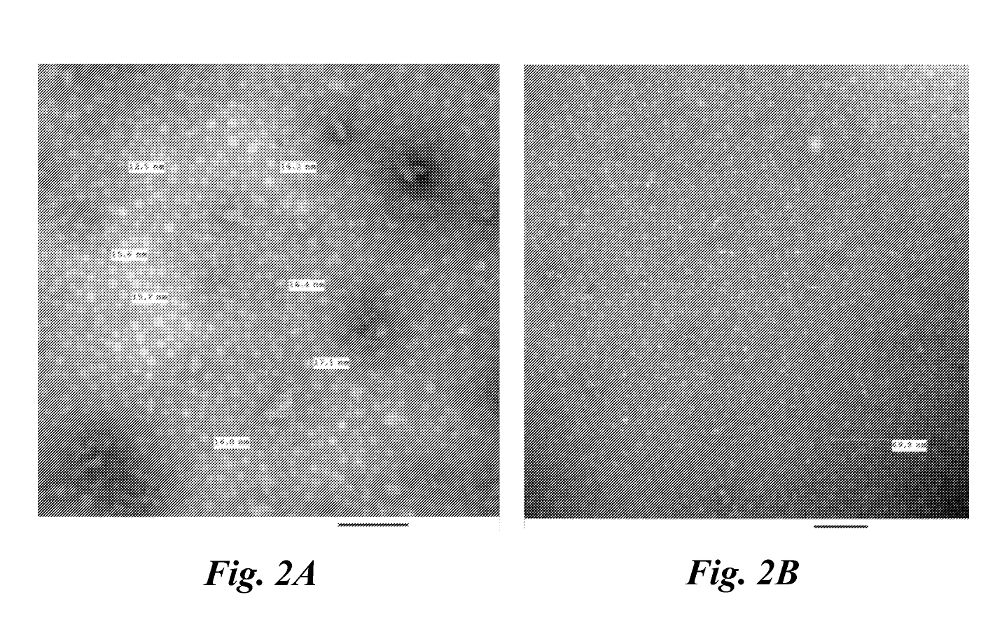

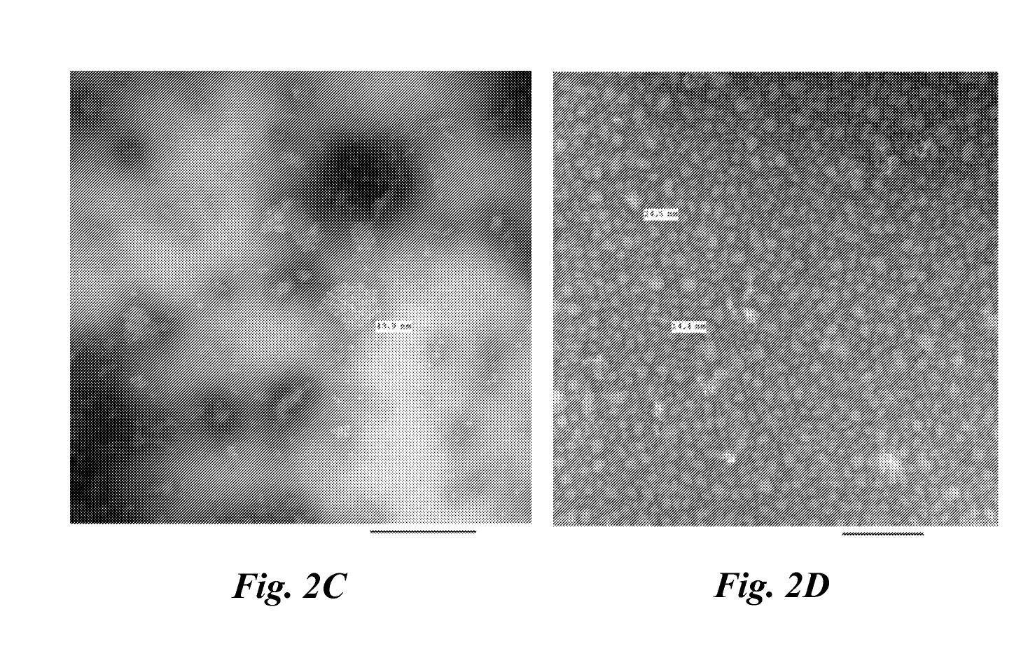

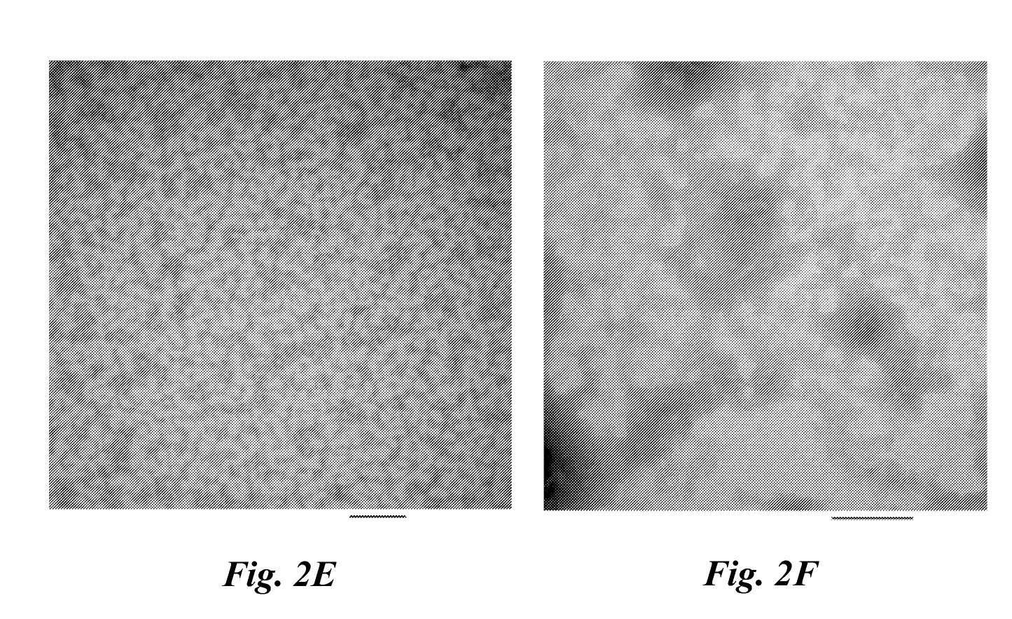

Morphologies of APNPs in different solutions were observed using a JEM-1010 Transmission Electron Microscope (JEOL, Tokyo, Japan). Samples in different aqueous conditions were prepared by dissolving the amphiphilic peptides in deionized water, phosphate-buffered saline, and acetic acid solutions at pH 2, 4, and 6. Next, a 5 μL aliquot of each sample was mounted on a 300-mesh copper grid (EM Sciences Ltd, North Vancouver, BC, Canada) and negatively stained by adding 5 μL of 1.5% aqueous phosphotungstic acid for 5 seconds. The excess liquid was removed carefully using filter paper. The images were captured by TEM at 40,000-50,000× magnification, operating at an accelerating voltage of 80 kV. The results are shown in FIGS. 2A-2D.

The TEM images showed that the peptide self-assembles into nanospheres during dialysis in deionized water and phosphate-buffered saline, with a mean diameter of 15.6 (range 10-20) nm at a concentration of 1.5 mg/mL (FIGS. 2A and 2B). The C18 aliphatic tail group serves as the driving force for the self-assembly behavior of APNPs, while the hydrophilic head group of the peptide functionalized by positively charged arginine-rich groups produces strong electrostatic interactions between adjacent molecules. Formation of APNPs with a spherical morphology was thus driven by the hydrophobic interactions between the tail groups and the electrostatic interactions between the head groups. The APNPs were found to aggregate when the peptide was dissolved in deionized water without sonication (FIG. 2C).

The estimated molecular length of the amphiphilic peptide C18GR7RGDS is 6.74 nm. Comparing the diameters of micelles measured in the TEM images and the theoretical molecular length, the micelle structure of APNPs is believed to be that of monolayer aggregates with solid hydrophobic cores.

Self-assembly of APNPs was pH dependent. At neutral pH in water, nanospherical aggregates formed, and these could still be observed at pH 6 in an acetic acid solution (FIG. 2D). However, at pH values of 2 and 4 (FIGS. 2E and 2F), only random cloud-like layers were observed, and the amphiphilic peptides did not self-assemble into nanospheres (micelles).

### Example 2

### Encapsulation of Curcumin in Amphphilic Peptide Nanoparticles

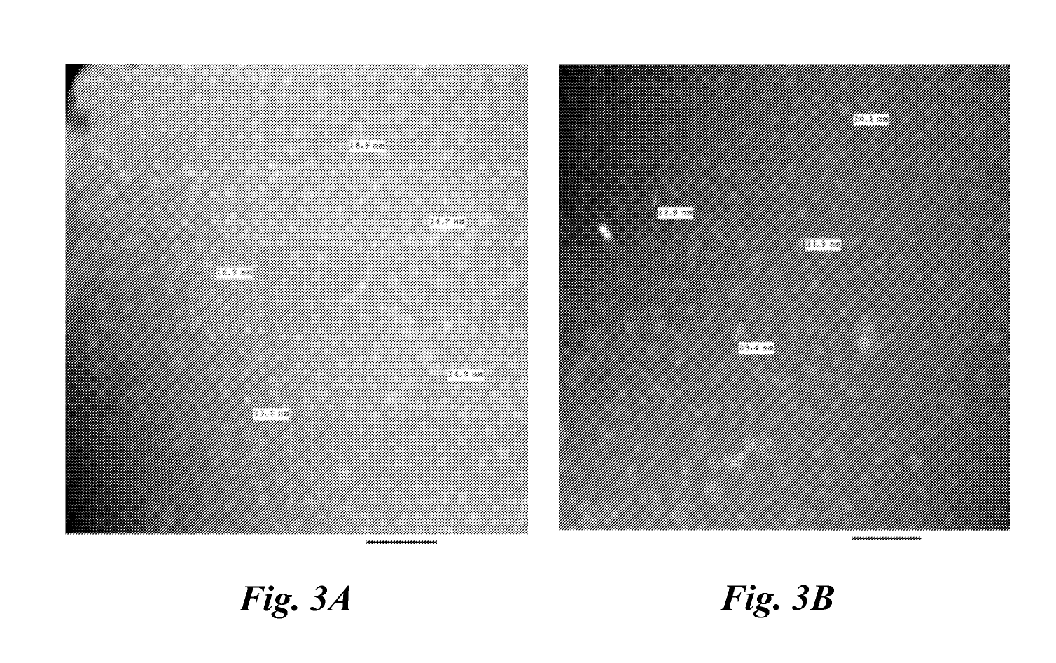

Curcumin-loaded APNPs were prepared by co-dissolution of curcumin with C18GR7RGDS at low pH followed by dialysis to raise the pH, which caused the self-assembly of APNPs and allowed the removal of monomeric (i.e., non-micellar) curcumin and C18GR7RGDS. First, curcumin was dissolved in 50% acetic acid and then added to a solution of dissolved amphiphilic peptide. In the mixture, the molar ratio of peptide to curcumin was 1:2. The mixture was then transferred to a dialysis tube having a dialysis membrane in the cap (molecular weight cutoff 1 kDa); the tube was inserted cap-down into a float and dialyzed against 800 mL of deionized water. The water was replaced by fresh deionized water every 4 hours in order to eliminate acetic acid and unloaded curcumin from the mixture in the dialysis tube. When the pH of the mixture was close to 7.0, the dialysis tubing was removed from the deionized water and the APNPs recovered. The morphology of the curcumin-loaded APNPs in the final solution were characterized by TEM as described in Example 1. The nanoparticles had a morphology similar to that of the pure APNPs of Example 1, but with larger diameters of about 18-30 nm (average diameter 22.8 nm, FIGS. 3A and 3B).

Co-dissolution of preformed APNPs made of pure C18GR7RGDS together with a curcumin solution in 50% acetic acid, followed by dialysis against deionized water, caused APNPs to reform into spherical nanostructures but with larger diameters than without curcumin. The solubility of curcumin increased significantly, and the orange-yellow curcumin-loaded APNP solution showed more stability and homogeneity than without loading into APNPs. Even after freeze-drying, the resulting powder could be dissolved easily and rapidly in water with retention of the previously loaded curcumin.

Drug-loaded nanoparticles had a morphology by TEM similar to that of the pure APNPs but with somewhat larger diameter. Thus, the self-assembly behavior was not significantly altered during the drug preparation procedure, and the pH-sensitive nanoparticles were able to form upon removal of acetic acid. Hydrophobic molecules such as curcumin could be entrapped and solubilized in the stearyl C18 aliphatic cores of the micelles through energetically favorable hydrophobic interactions, producing successful drug encapsulation in the aqueous APNP solution.

The amount of curcumin encapsulated in the APNPs was characterized by a standard curve showing a linear correlation between the known concentrations of curcumin in DMSO and the corresponding absorbance measured by ultraviolet-visible spectroscopy (SpectraMax M3, Molecular Devices, Sunnyvale, Calif., USA) at a wavelength of 430 nm (R2.0.98). Briefly, an aliquot of the curcumin-loaded APNP solution was lyophilized using a freeze-dryer (FreeZone 2.5 Plus, Labconco, Kansas City, Mo., USA). The dry powder was then dissolved in DMSO, and the concentration of curcumin was evaluated by correlating the absorption of this solution at 430 nm wavelength with a standard curve. The concentration of curcumin was evaluated three times for each sample. The average value of each triplicate was used to evaluate the curcumin encapsulation efficiency (EE %) and loading level (LL %), which were calculated by the following equations:

EE %=(wt drug encapsulated/wt drug added)×100%

LL %=(wt drug encapsulated/wt micelles)×100%

Compared to the same amount of a solid curcumin suspension in water (solubility less than 0.1 mg/ml), the resulting solution (EE %=8.4±2.5%, LL %=3.6±1.2%) showed significantly increased solubility of curcumin over its unaided solubility in water. Using the loading level of 3.6%, the molecular weight of C18GR7RGDS as 1850 Da, and that of curcumin as 368 Da, the loading was estimated to correspond to about 18% on a molar basis, or an average of six molecules of C18GR7RGDS to one molecule of curcumin. Moreover, this solution exhibited stability even after lyophilization. The lyophilized powder could be re-dissolved in water easily to reconstitute the micelles without loss of curcumin content or solubility.

### Example 3

### Characterization of Curumin-Loaded Amphiphilic Peptide Nanoparticles

Curcumin-loaded APNPs were prepared as described in Example 2. The composition and structure of the APNPs were characterized by zeta potential, IR spectroscopy, and X-ray diffraction.

The zeta potentials of pure APNPs (without curcumin) and curcumin-loaded APNPs were determined using a ZS90 Nanosizer (Malvern Instruments, Malvern, UK). Solutions containing 0.4 mg/mL of pure APNPs and curcumin-loaded APNPs were prepared in deionized water followed by sonication for 60 seconds at room temperature. The zeta potential of the nanoparticles was determined using 1 mL of each sample, each measured for ten preparations in triplicate.

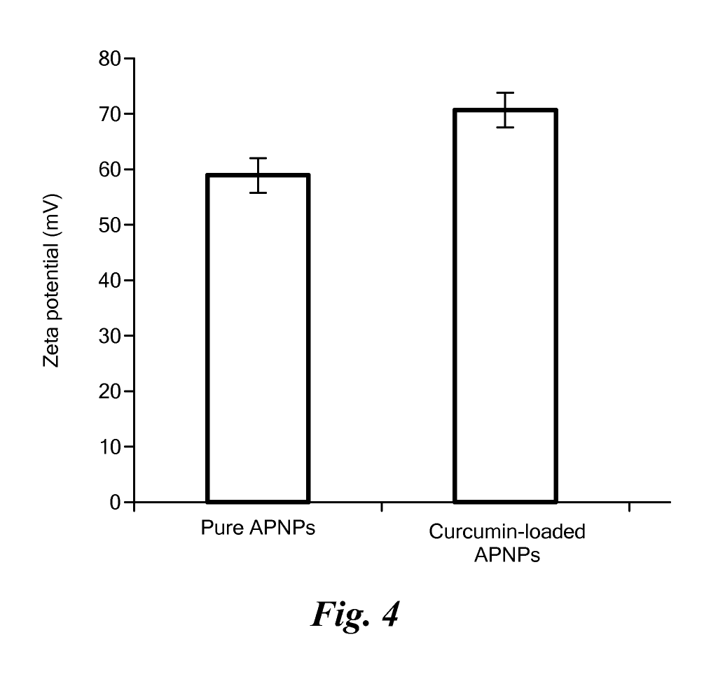

The measured average zeta potential of pure APNPs was +59±3.15 mV, while that of curcumin-loaded APNPs was +70.63±3.02 mV (FIG. 4). This result indicates that both pure and curcumin-loaded APNPs were stable in aqueous solution. The curcumin-loaded micelles have a higher zeta-potential, believed to result from the increased number of free peptide monomers aggregated to form stable micelles after drug loading. The positively charged micelles facilitate cellular uptake mediated by the negative membrane potential.

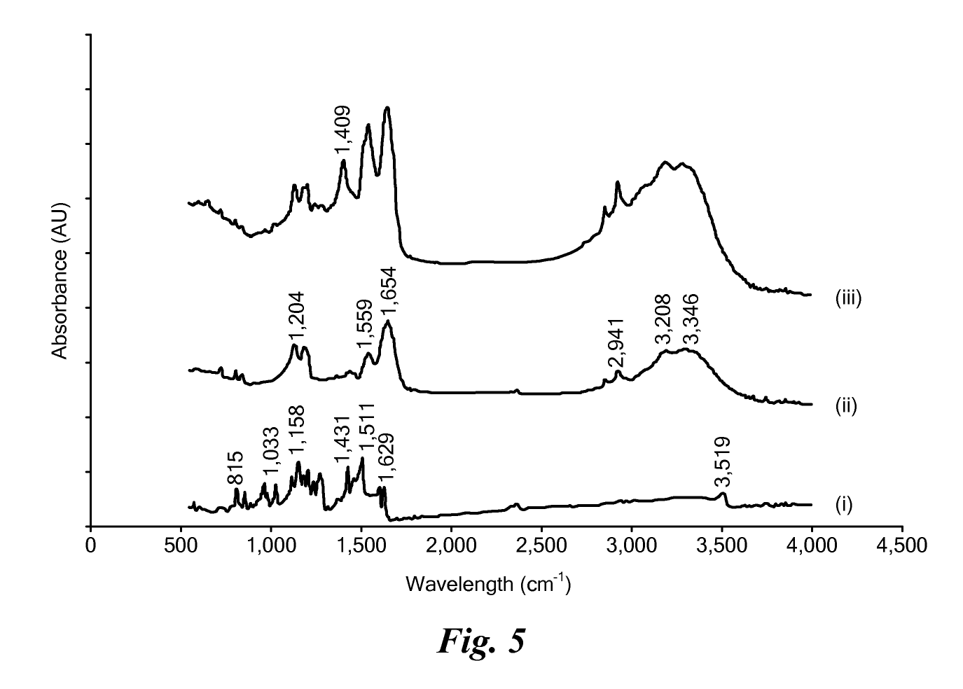

Fourier transform infrared (FT-IR) spectra of pure curcumin, C18GR7RGDS peptide powder, and lyophilized curcumin-loaded APNPs were obtained in order to analyze the chemical structure of these compounds and possible changes therein after drug loading of APNPs. Samples were analyzed using an FT-IR spectrometer (Vertex 70, Bruker Corporation, Billerica, Mass., USA) using the attenuated total reflectance method. The FT-IR spectra were collected in the wavelength range of 550-4,000 cm⁻¹ with a resolution of 2 cm⁻¹. The results are shown in FIG. 5.

In the spectra of plain curcumin, the bands that appeared in the ranges of 1,225-1,175 cm⁻¹ and 1,125-1,090 cm⁻¹, together with two additional weak bands in the ranges around 1,070-1,000 cm⁻¹, could represent the 1:2:4-substitution of the aromatic rings. The two C═C bonds conjugated with the neighborhood aromatic rings and C═O bonds could be characterized at 1,629 cm⁻¹ and 1,606 cm⁻¹, respectively. The hydroxyl group with intramolecular hydrogen bonds in the phenol groups could be characterized by the relatively weak absorption at 3,519 cm⁻¹.

In the spectra of pure C18GR7RGDS APNPs, the absorption at 1,654 cm⁻¹ could represent the amide I group, while the band at 1,560 cm⁻¹ could indicate the COON group in the amino acid sequence. In addition, the two wide bands at 3,400-3,300 cm⁻¹ could characterize the amine group of the arginine-rich structure. For the spectra of lyophilized curcumin-loaded APNPs, the bands appeared at a wavelength similar to that for pure APNPs, but the band at 1,409 cm⁻¹ could represent the OH deformation vibration on phenols. FT-IR spectra may suggest that the chemical structure of the amphiphilic peptide was not altered after drug loading since no significant band shifts were observed. Furthermore, most of the absorbance bands for curcumin could not be observed, except for the OH deformation vibration on the phenols. This indicates successful encapsulation of curcumin by APNPs, as curcumin molecules were shielded in the inner core of micelles, and the infrared radiation could not be transmitted through the encapsulated molecules.

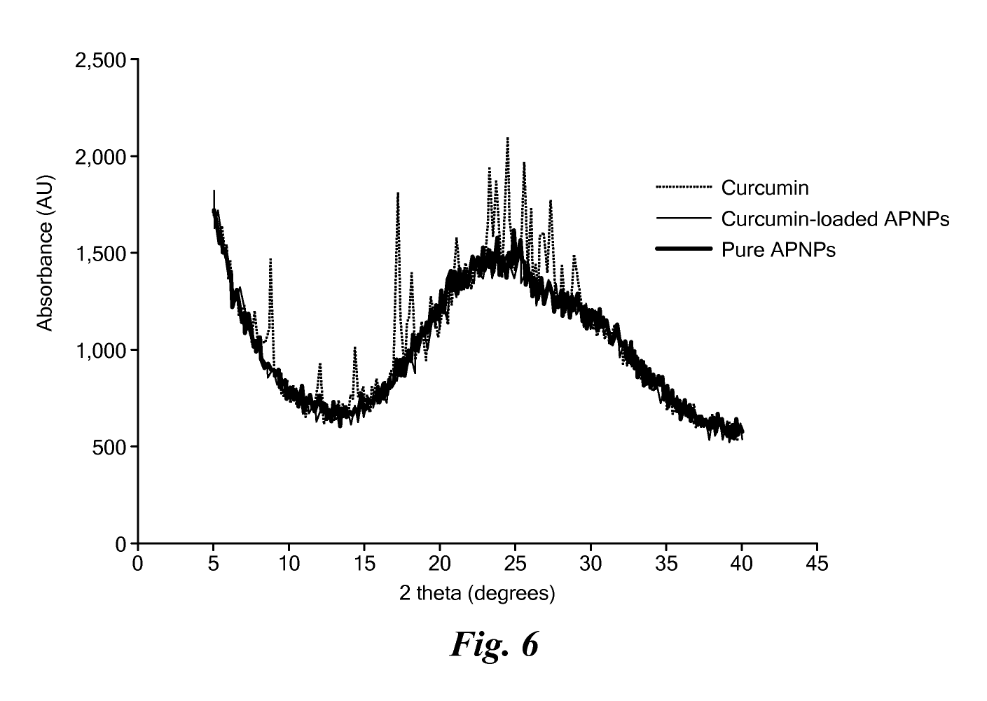

An X-ray diffraction (XRD) study was conducted to analyze the crystallographic structure of curcumin, pure APNPs, and lyophilized curcumin-loaded APNPs. Samples were analyzed using an X-ray diffractometer (Ultima IV, Rigaku Corporation, Tokyo, Japan) at a voltage of 40 kV, 44 mA, and 1.76 kW. The scanned angle was in the range of 5°≦2θ°≦40° and the scan rate was 3° per minute. The results are shown in FIG. 6.

In the XRD pattern for curcumin, a series of characteristic peaks could be observed in the range of 15°≦2θ°≦30°, representing the distinct crystalline structure of curcumin molecules. In contrast, pure APNPs may not have a characteristic crystalline structure since no peaks were evident in its XRD pattern. More importantly, the curcumin-loaded APNPs showed an XRD pattern similar to that of pure APNPs and did not show an observable crystalline structure. Disappearance of peaks characteristic of the crystalline structure of curcumin resulted from encapsulation by APNPs. The XRD pattern for the pure APNPs demonstrated that these molecules exist in a disordered crystalline structure or an amorphous structure. Thus, the XRD pattern of curcumin-loaded APNPs further confirmed successful drug encapsulation.

### Example 4

### Toxicity of Curcumin-Loaded APNPs Towards Osteosarcoma Cells

MG-63 osteosarcoma and noncancerous human healthy osteoblast cell lines were used to evaluate the cytotoxicity of plain curcumin suspended in phosphate-buffered saline, curcumin dissolved in DMSO, a solution of pure C18GR7RGDS APNPs, and a curcumin-loaded C18GR7RGDS APNP solution by the colorimetric MTT assay.

MG-63 osteosarcoma (CRL-1427) cells (American Type Culture Collection) were cultured in Eagle's Minimum Essential Medium supplemented with 10% fetal bovine serum and 1% penicillin/streptomycin, while healthy human osteoblasts (C-12760, PromoCell) were cultured in complete growth medium composed of osteoblast basal medium and osteoblast growth medium Supplement Mix. Both cell lines were incubated at 37° C. in a humidified incubator with an atmosphere of 95% oxygen and 5% CO₂. Cells were used at population doubling numbers less than 3. Eagle's Minimum Essential Medium was purchased from the American Type Culture Collection (Manassas, Va., USA), and osteoblast basal medium and osteoblast growth medium Supplemental Mix were purchased from PromoCell (Heidelberg, Germany). Methyl-thiazolyl-tetrazolium (MTT) dye solution was purchased from Promega (Madison, Wis., USA). 4′,6-diamidino-2-phenylindole (DAPI), and Atto Rho6G phalloidin were supplied by Sigma-Aldrich (St Louis, Mo., USA).

A confocal laser scanning microscope and a bright field microscope were used for a qualitative study of the cellular uptake of curcumin from curcumin-loaded APNPs. First, 1 mL each of the osteosarcoma cell line and the healthy human osteoblast cell line were seeded on a 24-well plate at a density of 2×10⁴ cells/mL. After 24 hours of incubation in 5% CO₂ and at 37° C., the cells were treated for 2 hours with 20 μM of curcumin encapsulated in APNPs or pure curcumin suspended in phosphate-buffered saline. The cells were then rinsed with phosphate-buffered saline three times to remove the unabsorbed curcumin. The qualitative uptake of curcumin was then monitored by bright field microscopy.

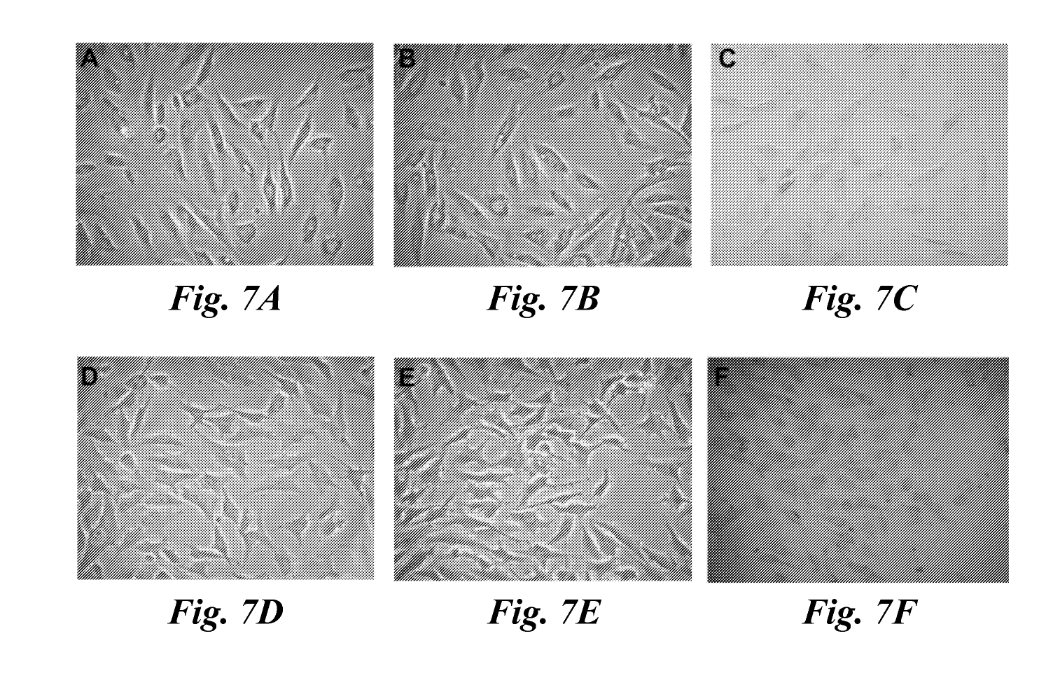

The osteosarcoma cells showed significantly higher uptake of curcumin than the normal human osteoblast cells in bright field microscopy images (FIGS. 7A-7F). In the samples treated only with plain curcumin suspended in phosphate-buffered saline, very small amounts of crystalline curcumin could be observed at the cell surface, but curcumin did not accumulate in the cytosol.

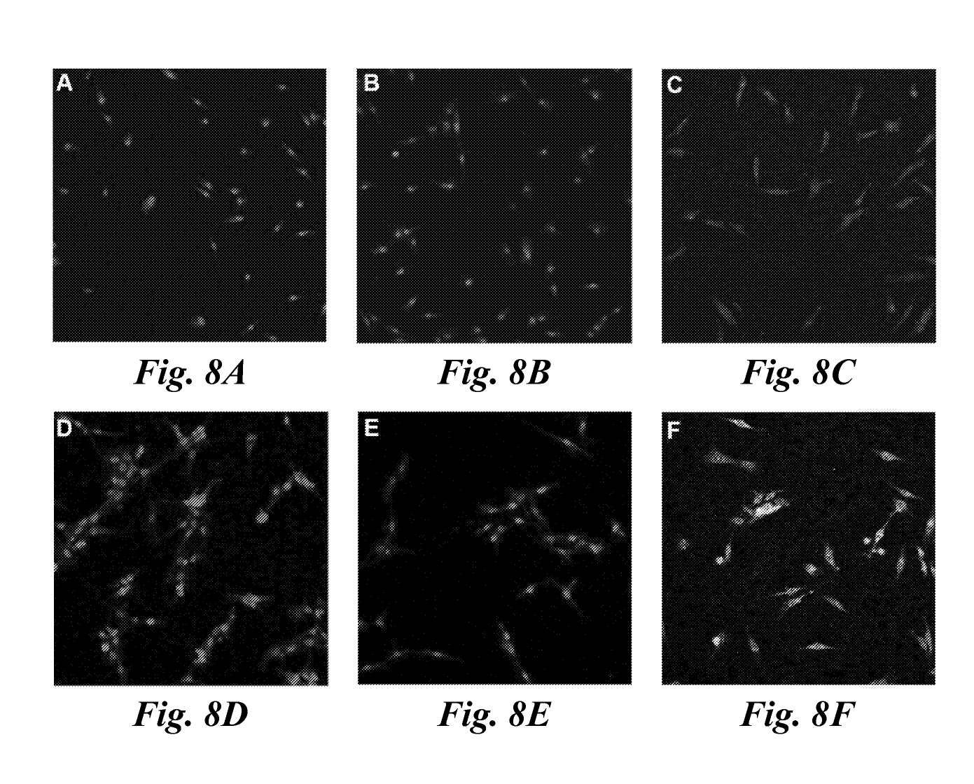

In another study, the nuclei of the cells were tracked by blue fluorescent DAPI staining using confocal microscopy (FIGS. 8A-8F), and the F-actin filaments of cells were stained with red fluorescent Rhodamine 6G. After 10 minutes of fixation by 10% formaldehyde solution and subsequent treatment with a 0.1% Triton X-100 solution for 10 minutes, the cells were stained with DAPI and Atto Rho6G phalloidin and observed using a Zeiss LSM710 laser scanning confocal microscope. The stained cells were then viewed for DAPI fluorescence (excitation 358 nm, emission 461 nm) and Atto Rho6G phalloidin fluorescence (excitation 525 nm, emission 560 nm), and curcumin uptake was observed using a fluorescein isothiocyanate filter (excitation 495 nm, emission 519 nm),¹⁰ Similar to the images taken by bright field microscopy, neither cell line showed detectable fluorescence of curcumin in the samples treated by plain curcumin. However, osteosarcoma cells treated with curcumin-loaded APNPs showed a strong green fluorescence, indicating that these cells accumulated significant amounts of curcumin into the cytosol. Normal human osteoblast cells showed only a weak green fluorescence in the cytosol.

These results demonstrated that curcumin-loaded APNPs could penetrate the surface membrane of osteosarcoma cells more efficiently and induce significantly higher cellular uptake than in human osteoblast cells. With an RGD-functionalized head group, the curcumin-loaded micelles are believed to selectively attach to the receptors of the overexpressed integrins on osteosarcoma cells, leading to more drug accumulation on the surface of the osteosarcoma cells than on the normal human osteoblast cells. Further, the positively-charged micelles can attach to carboxylate, sulfate, and phosphate groups on the cell surface by electrostatic interactions or hydrogen bonds, which favors macropinocytosis-meditated internalization of arginine-rich peptides. Hence, curcumin molecules are believed to internalize into the cytosol efficiently via the endosomal pathway from the cell surface membrane to the lysosome.

Next, the impact of APNP-delivered curcumin on sarcoma cell viability was investigated. 100 μL of the osteosarcoma and healthy osteoblast cell suspensions were seeded on a 96-well plate at 2×10³ cells/well (cell density 6,154 cells/cm²). After 24 hours of incubation in 5% CO₂ at 37° C. for attachment, the cells were treated with plain curcumin in phosphate-buffered saline, curcumin dissolved in DMSO, and a curcumin-loaded APNP solution containing different curcumin concentrations (3, 5, 10, 20, and 30 μM). For the cells treated with a solution of pure APNPs, the solution was prepared by the same co-dissolution and dialysis method as that used for the preparation of curcumin-loaded APNPs (see Example 2). Cells treated with medium only were used as a positive control. For the samples treated with curcumin dissolved in DMSO, cells treated with the same amount of DMSO (less than 0.5% v/v) were regarded as control samples. Serum-free medium was used in all samples to avoid interactions between the arginine-rich peptides and serum albumin.

The cells were treated for 24 hours. The medium was then removed from each well, and the cells were washed three times with phosphate-buffered saline. Next, 100 μL of cell culture medium and 15 μL of the MTT dye solution were added to each well, and the cells were incubated for 4 hours to allow the formation of formazan crystals. At the end of incubation, 100 μL of the MTT stop solution were added to each well. The 96-well plates were then tested using a spectrophotometer (SpectraMax M3, Molecular Devices) at a wavelength of 570 nm to obtain the optical density. Cell density was obtained from a standard curve expressing the linear correlation between different cell densities and optical densities (R²=0.98). Cell viability was expressed as the ratio of cell density in each sample to the cell density in the control sample.

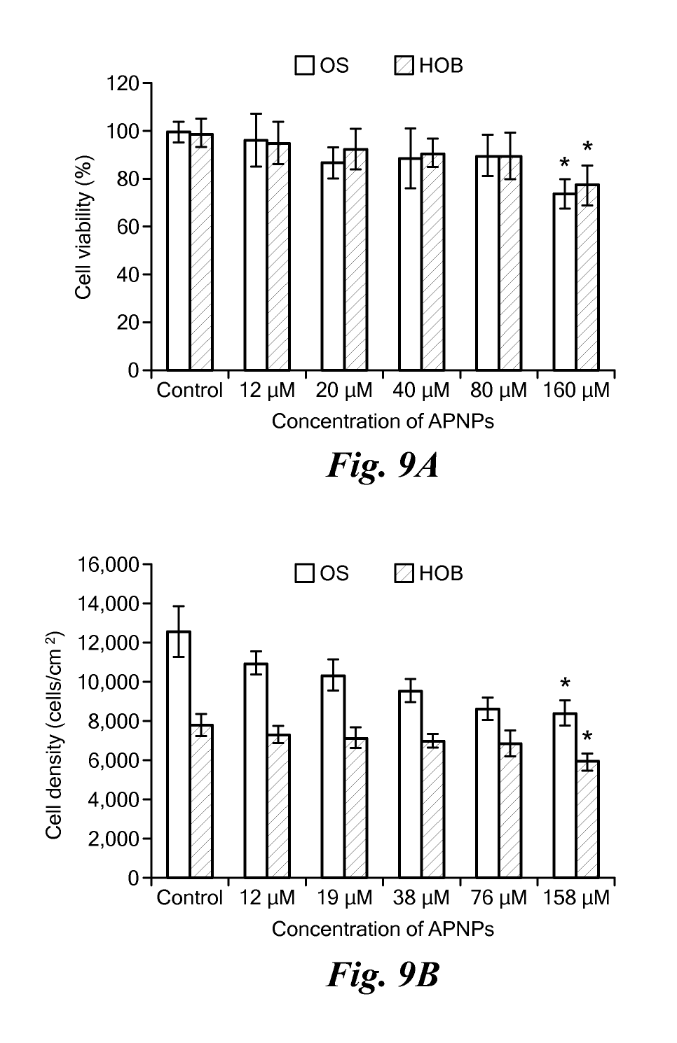

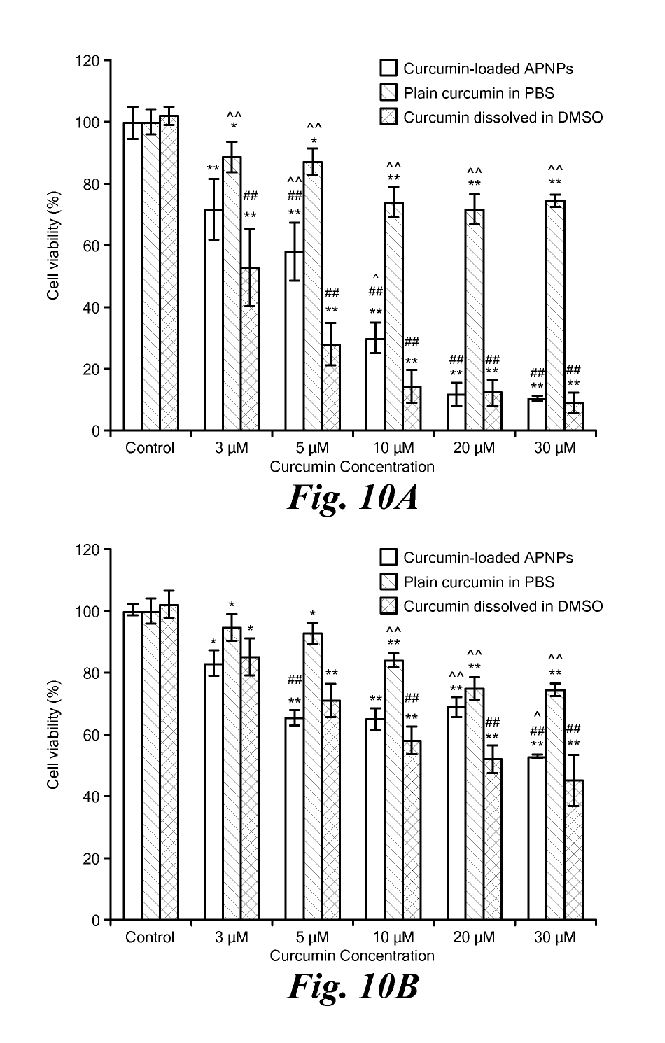

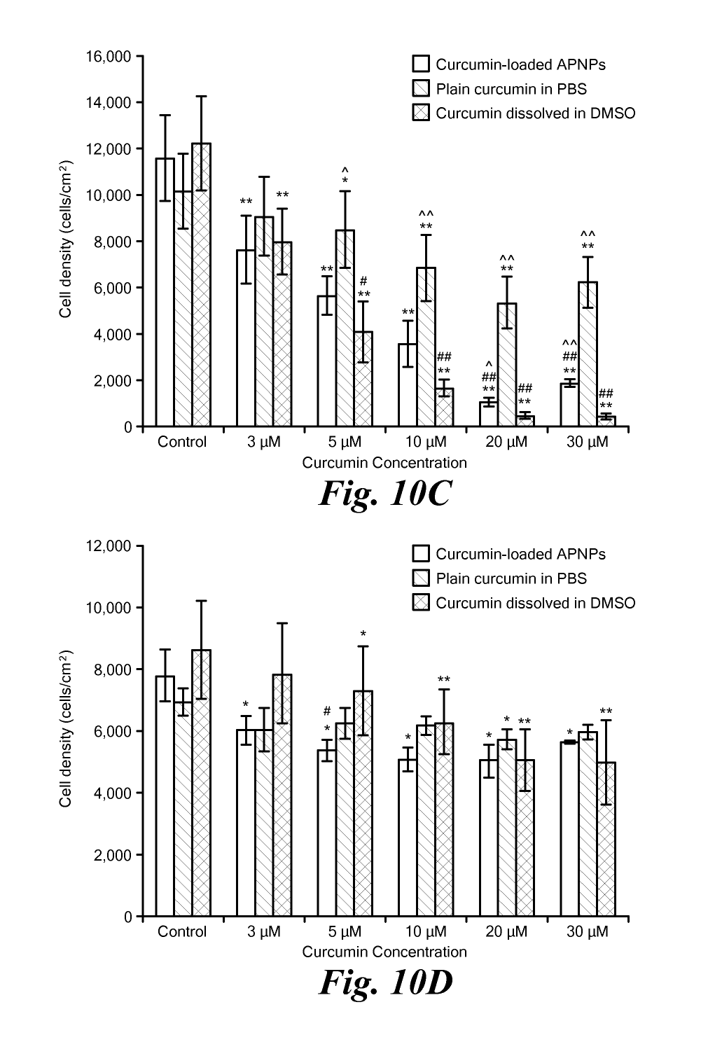

The pure C18GR7RGDS APNPs showed minor cytotoxicity in both the osteosarcoma cell line and the human osteoblast cell line at the highest concentration investigated (FIGS. 9A and 9B). The cytotoxicity of plain curcumin suspended in phosphate-buffered saline was insignificant for both cell lines (FIGS. 10A-10D), possibly reflecting low cellular uptake due to the low solubility of curcumin in aqueous solution. When dissolved in DMSO, curcumin was more cytotoxic to osteosarcoma cells at all concentrations investigated. More importantly, the curcumin-loaded APNPs showed significant selective reduction of viability in osteosarcoma cells. Compared with the curcumin/DMSO sample, the cytotoxicity of curcumin-loaded C18GR7RGDS APNPs was more selective for osteosarcoma cells in the concentration range of 20-30 μM (total curcumin concentration in the medium). At a curcumin concentration of 30 μM, the viability of osteosarcoma cells was as low as 15% after treatment with curcumin-loaded APNPs, whereas over 50% of human osteoblast cells were viable at this curcumin concentration. A 20 μM concentration of APNP-loaded curcumin appeared to be optimal, given that the viability of osteosarcoma was the minimum value at this concentration. This result confirms the targeting effects of the RGD peptide sequence on αvβ3 integrins, which are overexpressed on cancer cells, leading to more uptake of encapsulated drug.

### Example 5

### Bacteriostatic Effect of APNPs

The effect of pure C18GR7RGDS APNPs on bacterial growth and viability was investigated. C18GR7RGDS APNPs were prepared by dissolving C18GR7RGDS in sterile deionized water.

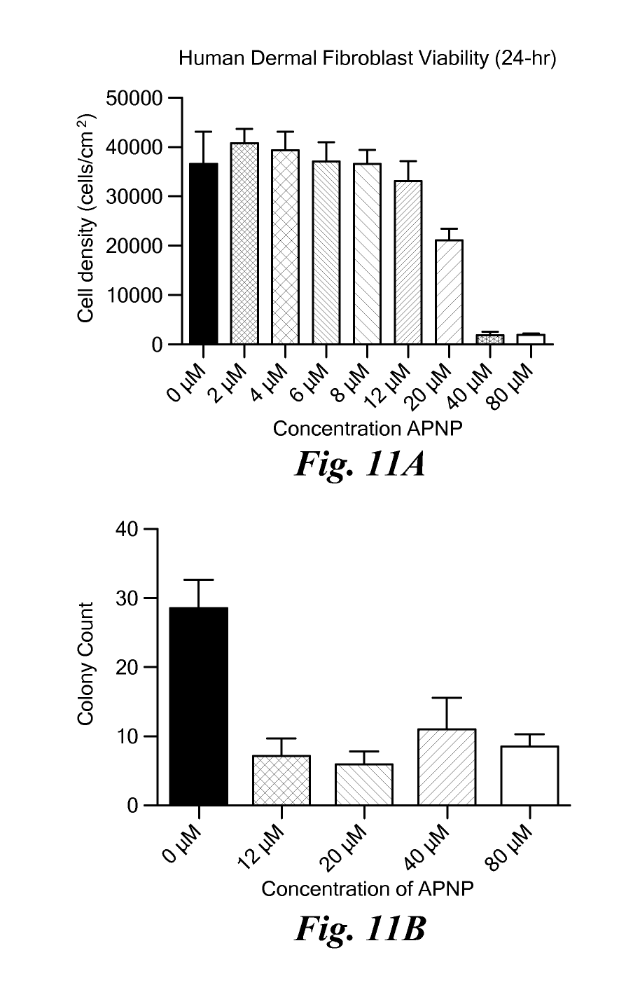

Human dermal fibroblasts (Lonza, CC-2511) were plated at a density of 10,000 cells/cm² in a 96-well plate and maintained in DMEM culture medium supplemented with 10% fetal bovine serum (FBS, Hyclone) and 1% penicillin/streptomycin (P/S, Hyclone). APNPs were added to the culture medium to achieve the indicated final concentration of C18GR7RGDS, and the cells were incubated for 24 hours prior to determination of cell density by MTS assay. The results are shown in FIG. 11A, and indicate that concentrations of C18GR7RGDS APNPs of 40 μM and above resulted in significant loss of cell viability as manifested by reduced density of living cells.

In a parallel experiment, the effect of pure C18GR7RGDS APNPs on Staphylococcus aureus (ATCC 12600) growth was determined. S. aureus cells were seeded at a density of 10⁵ CFU/ml in tryptic soy broth (TSB), and APNPs were added to liquid cultures of S. aureus to achieve the indicated final concentration. Growth was determined by plating aliquots of a dilution of the bacterial culture (10⁴) onto agar plates after 24 hours, incubating the plates for another 15 hours, and then counting the colonies. The results are shown in FIG. 11B, and indicate major loss of cell viability occurring between 0 and 12 μM of APNPs in the medium.

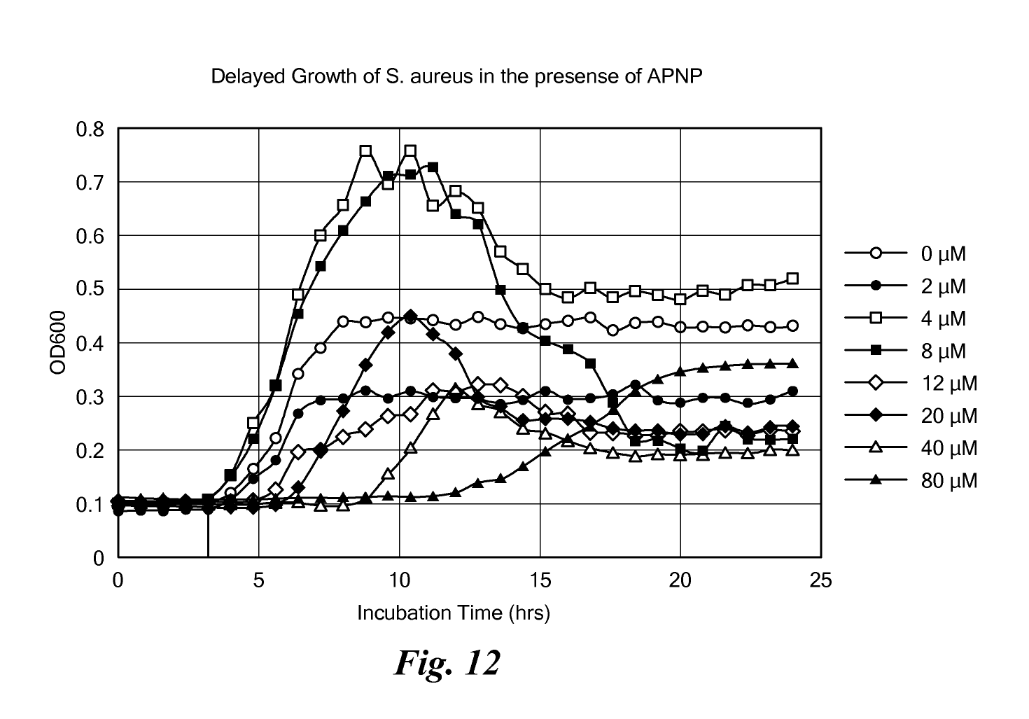

The effect of pure C18GR7RGDS APNPs on S. aureus growth kinetics also was investigated. The turbidity of liquid cultures of S. aureus with an initial concentration of 10⁵ CFU/ml was determined as the optical density at 600 nm wavelength as a function of time and in the presence of increasing concentrations of pure C18GR7RGDS APNPs. The results are shown in FIG. 12. Concentrations of APNPs as low as 2 μM showed inhibitory effects on S. aureus growth, observable either as a decrease in steady state turbidity achieved after 15-25 hours, or as an increase in the lag time to onset of exponential growth, with the latter effect being particularly significant at APNP concentrations of 12 μM and above.

As used herein, “consisting essentially of” does not exclude materials or steps that do not materially affect the basic and novel characteristics of the claim. Any recitation herein of the term “comprising”, particularly in a description of components of a composition or in a description of elements of a device, can be exchanged with “consisting essentially of” or “consisting of”.

While the present invention has been described in conjunction with certain preferred embodiments, one of ordinary skill, after reading the foregoing specification, will be able to effect various changes, substitutions of equivalents, and other alterations to the compositions and methods set forth herein.

This application claims the priority of U.S. Provisional Application No. 62/021,857 filed 08 Jul. 2014 and entitled “C18R7RGDS self-assembled amphiphilic peptide nanoparticles (APNPs) as a novel hydrophobic drug carrier in aqueous solution”, the whole of which is hereby incorporated by reference.

### REFERENCES

1. Strimpakos A S, Sharma R A. Curcumin: preventive and therapeutic properties in laboratory studies and clinical trials. Antioxid Redox Signal. 2008; 10(3):511-545.
2. Wilken R, Veena M S, Wang M B, Srivatsan E S. Curcumin: a review of anti-cancer properties and therapeutic activity in head and neck squamous cell carcinoma. Mol Cancer. 2011; 10:12.
3. Brennan P, O'Neill L A. Inhibition of nuclear factor KB by direct modification in whole cells—mechanism of action of nordihydroguaiaritic acid, curcumin and thiol modifiers. Biochem Pharmacol. 1998; 55(7):965-973.
4. Jobin C, Bradham C A, Russo M P, et al. Curcumin blocks cytokine-mediated NF-kappa B activation and proinflammatory gene expression by inhibiting inhibitory factor I-kappa B kinase activity. J Immunol. 1999; 163(6):3474-3483.
5. Choudhuri T, Pal S, Das T, Sa G. Curcumin selectively induces apoptosis in deregulated cyclin D1-expressed cells at G2 phase of cell cycle in a p53-dependent manner. J Biol Chem. 2005; 280(20):20059-20068.
6. Liu E, Wu J, Cao W, et al. Curcumin induces G2/M cell cycle arrest in a p53-dependent manner and upregulates ING4 expression in human glioma. J Neurooncol. 2007; 85(3):263-270.
7. Collins H M, Abdelghany M K, Messmer M, et al. Differential effects of garcinol and curcumin on histone and p53 modifications in tumour cells. BMC Cancer. 2013; 13:37.
8. Kumar A, Ahuja A, Ali J, Baboota S. Conundrum and therapeutic potential of curcumin in drug delivery. Crit Rev Ther Drug Carrier Syst. 2010; 27(4):279-312.
9. Tønnesen HH. Solubility, chemical and photochemical stability of curcumin in surfactant solutions. Pharmazie. 2002; 57(12):820-824.
10. Mohanty C, Sahoo SK. The in vitro stability and in vivo pharmacokinetics of curcumin prepared as an aqueous nanoparticulate formulation. Biomaterials. 2010; 31(25):6597-6611.
11. Chen J X, Wang H Y, Quan C Y, Xu X D, Zhang X Z, Zhuo R X. Amphiphilic cationic lipopeptides with RGD sequences as gene vectors. Org Biomol Chem. 2010; 8(14):3142-3148.

1. A nanoparticulate carrier formulation for a hydrophobic drug, the formulation comprising
a plurality of amphiphilic peptide molecules, each molecule comprising a hydrophobic portion covalently linked to a positively charged hydrophilic portion; wherein the molecules are assembled into a plurality of substantially spherical nanoparticles in an aqueous medium having a nonacidic pH; each nanoparticle comprising a hydrophobic core; and
a plurality of hydrophobic drug molecules embedded in the hydrophobic core of the nanoparticles;
wherein the hydrophobic drug is solubilized in the aqueous medium of the formulation at a higher concentration than a solubility limit of the hydrophobic drug alone in the aqueous medium; and
wherein the nanoparticles are capable of delivering the drug to the interior of a mammalian cell.

2. The nanoparticulate carrier formulation of claim 1, wherein said nonacidic pH is greater than about 4.

3. The nanoparticulate carrier formulation of claim 2, of wherein the nanoparticles reversibly dissociate at a pH of about 4 or less and assemble at a pH greater than about 4.

4. The nanoparticulate carrier formulation of claim 1, wherein the molar ratio of amphiphilic peptide molecules to hydrophobic drug molecules is from about 2:1 to about 10:1.

5. The nanoparticulate carrier formulation of claim 1, wherein the hydrophobic portion comprises one or more straight or branched chain alkyl groups, cycloalkyl groups, aromatic hydrocarbons, or a combination thereof.

6. The nanoparticulate carrier formulation of claim 5, wherein the hydrophobic portion comprises one or more C8 to C22 alkyl groups.

7. The nanoparticulate carrier formulation of claim 6, wherein the hydrophobic portion consists of a single C18 alkyl group.

8. The nanoparticulate carrier formulation of claim 1, wherein the hydrophilic portion comprises two or more amino acids capable of bearing a positive charge at a physiological pH.

9. The nanoparticulate carrier formulation of claim 8, wherein the hydrophilic portion comprises five or more amino acid residues selected from arginine, lysine, and mixtures thereof.

10. The nanoparticulate carrier formulation of claim 1, wherein the hydrophilic portion comprises a targeting moiety.

11. The nanoparticulate carrier formulation of claim 10, wherein the targeting moiety comprises an RGD peptide, an antibody, an aptamer, or a ligand for a cell surface receptor.

12. The nanoparticulate carrier formulation of claim 1, wherein the amphiphilic peptide has a log P value of 1 or more.

13. The nanoparticulate carrier formulation of claim 1, wherein the amphiphilic peptide has a log D value of 1 or more at pH 7.4.

14. The nanoparticulate carrier formulation of claim 1, wherein the amphiphilic peptide is C18GR7RGDS (SEQ ID NO:1).

15. The nanoparticulate carrier formulation of claim 1, wherein the nanoparticles bind to a cell surface.

16. The nanoparticulate carrier formulation of claim 1, wherein the nanoparticles release the hydrophobic drug molecules into an intracellular compartment having a pH of 4 or less.

17.-21. (canceled)

22. The nanoparticulate carrier formulation of claim 1 which is present in lyophilized form.

23.-24. (canceled)

25. A method of making the nanoparticulate carrier formulation of claim 1, the method comprising the steps of:
(a) providing an aqueous medium comprising a positively charged amphiphilic peptide, wherein the aqueous medium has an acidic pH and the amphiphilic peptide is in a dissociated state;
(b) adding a hydrophobic drug to the aqueous medium; and
(c) raising the pH of the aqueous medium, whereby the amphiphilic peptide forms nanoparticles having a hydrophobic core, and whereby the hydrophobic drug becomes embedded in the hydrophobic core of the nanoparticles.

26. The method of claim 25, further comprising:
(d) removing nonembedded hydrophobic drug from the aqueous suspension.

27. The method of claim 26, further comprising:
(e) lyophilizing the carrier formulation.

28. The method of claim 25, further comprising, prior to step (a):
(a0) providing an aqueous medium comprising a positively charged amphiphilic peptide, wherein the aqueous medium has a nonacidic pH and the amphiphilic peptide is associated in the form of nanoparticles; and
(a00) lowering the pH of the aqueous medium to an acidic pH, whereby the nanoparticles dissociate.

29.-35. (canceled)

36. A method of administering a hydrophobic drug, the method comprising administering to a subject in need thereof the nanoparticulate carrier formulation of claim 1, whereby the hydrophobic drug is delivered to an intracellular site in the subject.

37.-39. (canceled)

40. A method of inhibiting the growth and/or replication of bacteria, the method comprising contacting the bacteria with a plurality of amphiphilic nanoparticles; wherein the amphiphilic nanoparticles comprise a plurality of associated amphiphilic peptide molecules, each peptide molecule comprising a hydrophobic portion covalently linked to a positively charged hydrophilic portion; wherein the nanoparticles are substantially spherical and have a positively charged surface and a hydrophobic core; wherein the nanoparticles are formulated in an aqueous medium having a nonacidic pH; whereby the growth and/or replication of the bacteria are inhibited.

41.-43. (canceled)

44. A cosmetic composition capable of inhibiting the growth or replication of bacteria in or on skin; wherein the composition comprises a plurality of amphiphilic nanoparticles; wherein the amphiphilic nanoparticles comprise a plurality of associated amphiphilic peptide molecules, each peptide molecule comprising a hydrophobic portion covalently linked to a positively charged hydrophilic portion; wherein the nanoparticles are substantially spherical and have a positively charged surface and a hydrophobic core; wherein the composition is formulated in an aqueous medium having a nonacidic pH.

45.-47. (canceled)

48. A matrix for cell attachment, the matrix comprising an association of amphiphilic peptide molecules, each amphiphilic peptide molecule comprising a hydrophobic portion covalently linked to a positively charged hydrophilic portion; wherein the molecules are assembled into a matrix, wherein the hydrophobic portions and the hydrophilic portions of the peptide molecules are associated in the matrix.

49.-51. (canceled)

52. A medical implant comprising the matrix of claim 48.

53. (canceled)
